SEQUENCE LISTING (100 sequences)

SEQ ID NO: 1 - DNA, 801 bp, Artificial Sequence
  hybrid
atgaaaacatttgccgcatatgtaattatcgcgtgtctttcgagtacggcattagctagttcaattacagaaaatacgttttggaacaaagagttctctgccgaagccgtcaatggtgttttcgtgctttgtaaaagtagcagtaaatcctgcgctaccaataacttagctcgtgcatcaaaggaatatcttccagcatcaacatttaagatccccaacgcaattatcggcctagaaactggtgtcataaagaatgagcatcagattttcaaatgggacggaaaaccaagagccatgaaacaatgggaaagagacttgagcttaagaggggcaatacaagtttcagcggttcccgtatttcaacaaatcgccagagaagttggcgaagtaagaatgcagaaatatcttaaaaaattttcatatggtaaccagaatatcagtggcggcattgacaaattctggttggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 2 - protein, 266 aa, Artificial Sequence
  hybrid
MKTFAAYVIIACLSSTALASSITENTFWNKEFSAEAVNGVFVLCKSSSKSCATNNLARASKEYLPASTFKIPNAIIGLETGVIKNEHQIFKWDGKPRAMKQWERDLSLRGAIQVSAVPVFQQIAREVGEVRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 3 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcagattttcaaatgggacggaaagccaagagccatgaaacaatgggaaagagacttgaccttaagaggggcaatacaagtttcagcggttcccgtatttcaacaaatcgccagagaagttggcgaagtaagaatgcagaaatatcttaaaaaattttcatatggtaaccagaatatcagtggtggcattgacaaattctggtcggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 4 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQIFKWDGKPRAMKQWERDLTLRGAIQVSAVPVFQQIAREVGEVRMQKYLKKFSYGNQNISGGIDKFWSEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 5 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcagggaaagtgagggcatcattggtggctaa

SEQ ID NO: 6 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNESKLPLRKSIPTKIRESEGIIGG

SEQ ID NO: 7 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcaccaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaaggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 8 - protein, 266 aa, Artificial Sequence
  hybrid
MKTFAAYVIIACLSSTALAGSITENTSWNKEFSAEAVNGVFVLCKSSSKSCATNDLARAPKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 9 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatggacaacgaaagtaagttgccgctaagaaaatccattcccaccaaaatcatggaaagtgagggcatcattggtggctaa

SEQ ID NO: 10 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDMDNESKLPLRKSIPTKIMESEGIIGG

SEQ ID NO: 11 - DNA, 801 bp, Artificial Sequence
  hybrid
atgaaaacatttgccgcatatgtaattactgcgtgtctttcaagtacggcattagctagttcaattacagaaaatacgttttggaacaaagagttctctgccgaagccgtcaatggtgttttcgtgctttgtaaaagtagcagtaaatcctgcgctacctataacttagctcgtgcatcaaaggaatatcttccagcatcaacatttaagatccccaacgcaattatcggcctagaaactggtgtcataaagaatgagcatcagattttcaaatgggacggaaagccaagagccatgaaacaatgggaaagagacttgagcttaagaggggcaatacaagtttcagcggttcccgtatttcaacaaatcgccagagaagttggcgaagtaagaatgcagaaatatcttaaaaaattttcatatggtaaccagaatatcagtggtggcattgacaaattctggttggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 12 - protein, 266 aa, Artificial Sequence
  hybrid
MKTFAAYVITACLSSTALASSITENTFWNKEFSAEAVNGVFVLCKSSSKSCATYNLARASKEYLPASTFKIPNAIIGLETGVIKNEHQIFKWDGKPRAMKQWERDLSLRGAIQVSAVPVFQQIAREVGEVRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 13 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaggaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaatcagctaatagtaaaagaggctttggtaacggaggctgcgcctgaatatcttgtgcattcaaaaactggtttttctggtgtgggaactgagtcaaatcctggtgtcgcatggtgggttgggtgggttgagaaggagacagaggtttactttttcgcatttaacatggatatagacaacgaaaataagttgccgctaagaaaattcattcccaccaaaatcatggcaagtgagggcatcattggtggctaa

SEQ ID NO: 14 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIRNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNENKLPLRKFIPTKIMASEGIIGG

SEQ ID NO: 15 - DNA, 804 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagagactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggcaatagttacagaagcaactccagaatatatagttcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcatcattggtggctaa

SEQ ID NO: 16 - protein, 267 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEAIVTEATPEYIVHSKTGFSGVGTESNPGVAWWVGWVEKETEVYFFAFNMDIDNESKLPLRKSIPTKIMESEGIIIGG

SEQ ID NO: 17 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggtagagaaaggaactgaggtttactttttcgcctttagcatggatatagacaacgaaagtaagttgccgctaagaaaatccattcccaccaaaatcatggaaagtgagggcatcattggtggctaa

SEQ ID NO: 18 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKGTEVYFFAFSMDIDNESKLPLRKSIPTKIMESEGIIGG

SEQ ID NO: 19 - DNA, 804 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgagagtaaattgccgtcaagaaaatccatttcaacgaaaatcatggcaagtgaaggcatcatcattggtggctaa

SEQ ID NO: 20 - protein, 267 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNESKLPSRKSISTKIMASEGIIIGG

SEQ ID NO: 21 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtgagaatgcagaaataccttaaaaaattttcctatggcagccagaatatcagtggtggcattgacaaattctggttggaagaccagcttagaatttccgcagttaatcaagtggagtctctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcattggtggctaa

SEQ ID NO: 22 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVESLESLYLNKLSASKENQLIVKEALVTEAAPEYLVHSKTGFSGVGTESNPGVAWWVGWVEKETEVYFFAFNMDIDNESKLPLRKSIPTKIMESEGIIGG

SEQ ID NO: 23 - DNA, 804 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagaggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcatcattggtggctaa

SEQ ID NO: 24 - protein, 267 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQEEFLESLYLNKLSASKENQLIVKEALVTEAAPEYLVHSKTGFSGVGTESNPGVAWWVGWVEKETEVYFFAFNMDIDNESKLPLRKSIPTKIMESEGIIIGG

SEQ ID NO: 25 - DNA, 800 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgagcttaagaggggcaatacaagtttcagctgttcccgtatttcaacaaatcgccagagaagttggcgaagtaagaatgcagagataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgggaaggagacagaggtttactttttcgcctttaacatggatatagacaacgaaagtaagttgccgctaagaaatccattcccaccaaaatcatggaaagtgagggcatcattggtggctaa

SEQ ID NO: 26 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLSLRGAIQVSAVPVFQQIAREVGEVRMQRYLKKFSYGSQNISGGIDKFWLEDQLRISAVNQVEFLESLYLNKLSASKENQLIVKEALVTEAAPEYLVHSKTGFSGVGTESNPGVAWWVGWVGKETEVYFFAFNMDIDNESKLPLRKSIPTKIMESEGIIGG

SEQ ID NO: 27 - DNA, 802 bp, Artificial Sequence
  hybrid
atgaaaacatttgccgcatatgtaattactgcgtgtctttcaagtacggcattagctagttcaattacagaaaatacgttttggaacaaagagttctctgccgaagccgtcaatggtgttttttcgtgctttgtaaaagtagcagtaaatcctgcgctaccaataacttagctcgtgcatcaaaggaatatcttccagcatcaacatttaagatccccaacgcaattatcggcctagaaactggtgtcataaagaatgagcatcaggttttcaaatgggacggaaagccaagagccatgaaacaatgggaaagagacttgagcttaagaggggcaatacaagtttcagcggttcccgtatttcaacaaatcgccagagaagttggcgaagtaagaatgcagaaatatcttaaaaaattttcatatggtaaccagaatatcagtggtggcattgacaaattctggttggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaatcatggcaagtgagggcatcattggtggctaa

SEQ ID NO: 28 - protein, 266 aa, Artificial Sequence
  hybrid
MKTFAAYVITACLSSTALASSITENTFWNKEFSAEAVNGVFVLCKSSSKSCATNNLARASKEYLPASTFKIPNAIIGLETGVIKNEHQVFKWDGKPRAMKQWERDLSLRGAIQVSAVPVFQQIAREVGEVRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 29 - DNA, 801 bp, Artificial Sequence
  hybrid
atgaaaacatttgccgcatacgtaattactgcgtgtctttcaagtacggcattagctagttcaattacagaaaatacgttttggaacaaagagttctctgccgaagccgtcaatggtgttttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcagattttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcatatggtaaccagaatatcagtggtggcattgacaaattctggttggagggtcagcttagaattcccgcagttaatcaagtggagtttctagagtctctatttttaaataaattgtcagcatcaaaagaaaatcagctaatagtaaaagaggctttggtaacggaggcggcacctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 30 - protein, 266 aa, Artificial Sequence
  hybrid
MKTFAAYVITACLSSTALASSITENTFWNKEFSAEAVNGVFVLCKSSSKSCATNDLARASKEYLPASTFKIPNAIIGLETGVIKNEHQIFKWDGKPRAMKQWERDLTLRGAIQVSAVPVFQQIAREVGEVRMQKYLKKFSYGNQNISGGIDKFWLEGQLRIPAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 31 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcattggtggctaa

SEQ ID NO: 32 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNESKLPLRKSIPTKIMESEGIIGG

SEQ ID NO: 33 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaacatctagtgcattcaaaaactggtttttctggtgtgggaactgagtcaaatcctggtgtcgcatggtgggttgggtgggttgagaaggagacagaggtttactttttcgcctttaacatggacatagacaacgaaagtaagttgccgctaagaaaatccattcccaccaaaatcatggaaagtgagggcatcattggtggctaa

SEQ ID NO: 34 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEHLVHSKTGFSGVGTESNPGVAWWVGWVEKETEVYFFAFNMDIDNESKLPLRKSIPTKIMESEGIIGG

SEQ ID NO: 35 - DNA, 804 bp, Artificial Sequence
  hybrid
atgaaaacatttgccgcatatttaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcatcattggtggctta

SEQ ID NO: 36 - protein, 267 aa, Artificial Sequence
  hybrid
MKTFAAYLIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFAFNMDIDNESKLPLRKSIPTKIMESEGIIIGG

SEQ ID NO: 37 - DNA, 804 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttctgctgttcccgtatttcaacaaatcgccagagaagttggcgaagtaagaatgcagaaataccttaaaaaattttcctatggcagccagaatatcagtggtggcattgacaaattctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagactgaggtttacttttttgcttctaacatggacatagacaatgagagtaaattgccgtcaagaaaatccatttcaacgaaaatcatggcaagtgaaggcatcatcattggtggctaa

SEQ ID NO: 38 - protein, 267 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGSQNISGGIDKFWLEDQLRISAVNQVEFLESLYLNKLSASKENQLIVKEALVTEAAPEYLVHSKTGFSGVGTESNPGVAWWVGWVEKETEVYFFASNMDIDNESKLPSRKSISTKIMASEGIIIGG

SEQ ID NO: 39 - DNA, 801 bp, Pseudomonas. aeruginosa
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaataccttaaaaaattttcctatggcagccagaatatcagtggtggcattgacaaatcctggttggaagaccagcttagaatttccgcagttaatcaagtggagtttctagagtctctatatttaaataaattgtcagcatctaaagaaaaccagctaatagtaaaagaggctttggtaacggaggcggcacctgaatatctagtgcattcaaaaactggtttttctggtgtgggaactgagtcaaatcctggtgtcgcatggtgggttgggtgggttgagaaggagacagaggtttactttttcgcctttaacatggatatagacaacgaaagtaagttgccgctaagaaaatccattcccaccaaaatcatggaaagtgagggcatcattggtggctaa

SEQ ID NO: 40 - DNA, 801 bp, Escherichia coli
atgaaaacatttgccgcatatgtaattactgcgtgtctttcaagtacggcattagctagttcaattacagaaaatacgttttggaacaaagagttctctgccgaagccgtcaatggtgttttcgtgctttgtaaaagtagcagtaaatcctgcgctaccaataacttagctcgtgcaccaaaggaatatcttccagcatcaacatttaagatccccaacgcaattatcggcctagaaactggtgtcataaagaatgagcatcagattttcaaatgggacggaaaaccaagagccatgaaacaatgggaaagagacttgagcttaagaggggcaatacaagtttcagcggttcccgtatttcaacaaatcgccagagaagttggcgaagtaagaatgcagaaatatcttaaaaaattttcatatggtaaccagaatatcagtggcggcattgacaaattctggtcggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 41 - DNA, 804 bp, Pseudomonas aeruginosa
atgaaaaccatagccgcatacttagttacttcctgtttttcaagcaccgcgctctcaaagtctatttctgaaaatttggtgtggaataaagaattttctagtgaatccgtacatggcgtttttgtactttgtaaaagtagtagcaattcctgtactacaaataatgcggcacgtgcatctacagcctatattccagcatcaacattcaaaattcctaatgctctaataggtcttgaaaccggcgccataaaagatgaacggcagattttcaaatgggacggcaagcccagagccatgaaacaatgggaaaaagacttaaggctaaggggcgctatacaggtttctgcggttccggtatttcaacaaattgccagagaagttggcgaaatgagaatgcaaagatatcttaacctgttttcatacggtaacgccaatatagggggaggcattgacaaattctggctagagggtcagcttagaatcccagcattcaatcaagataaatctttagagtcgctcttcctgaataatttgccagcatcaaaagcaaatcaactaatagtaaaagaggcaatagttacagaagctacgccagaatatattgttcattcaaaaactgggtattccggtgttggcacagaatcaagtcctggtgtcgcttggtgggttggttgggtagggaaaggagctgaggtttacttttttgcatttaacatggacatagacaatgagaataaattgccgtcaagaaaatccatttcaacgaaaatcatggcaagtgaaggcatcatcattggtggctaa

SEQ ID NO: 42 - DNA, 827 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacattcaaaattcctaatgctctaataggtcttgaaaccggcgccataaaagatgaacggcaggttttcaaatgggacggcaagcccagagccatgaagcaatgggaaaaagacttaaagctaaggggcgctatacaggtttctgctgttccggtatttcaacaaattgccagagaagttggcgaaataagaatgcaaaaataccttaacctgttttcatacggcaacgccaatatagggggaggcattgacaaattctggctagaaggtcagcttagaatctcagcattcaatcaagttaaatttttagagtcgctctacctgaataatttgccagcatcaaaagcaaaccaactaatagtaaaagaggcaatagttacagaagcaactccagaatatatagttcattcaaaaactgggtattccggtgttggcacagaatcaagtcctggtgtcgcttggtgggttggttgggtagagaaaggaactgaggtttacttttttgcttttaacatggacatagacaatgagagtaaattgccgtcaagaaaatccattcccaccaaaatcatggcaagtgagggcatcattggtggctaagctgtgaagatcccagcaaaggctta

SEQ ID NO: 43 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNALIGLETGAIKDERQVFKWDGKPRAMKQWEKDLKLRGAIQVSAVPVFQQIAREVGEIRMQKYLNLFSYGNANIGGGIDKFWLEGQLRISAFNQVKFLESLYLNNLPASKANQLIVKEAIVTEATPEYIVHSKTGYSGVGTESSPGVAWWVGWVEKGTEVYFFAFNMDIDNESKLPSRKSIPTKIMASEGIIGG

SEQ ID NO: 44 - DNA, 801 bp, Artificial Sequence
  hybrid
atgaaaacatttgccgcatatttagttctcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggtctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaaataagaatgcagaaatatcttaaaaaattttcatatggtaaccagaatatcagtggtggcattgacaaattctggctagaaggtcagcttagaatctcagcattcaatcaagttaaatttttagagtcgctctacctgaataatttgccagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 45 - protein, 266 aa, Artificial Sequence
  hybrid
MKTFAAYLVLACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEIRMQKYLKKFSYGNQNISGGIDKFWLEGQLRISAFNQVKFLESLYLNNLPASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 46 - DNA, 801 bp, Artificial Sequence
  hybrid
atgaaaacatttgccgcatatgtaattatcgcgtgtctttcgagtacggcattagctagttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaaataagaatgcagaaatatcttaaaaaattttcatatggtaaccagaatatcagtggtggcattgacaaattctggttggagggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 47 - protein, 266 aa, Artificial Sequence
  hybrid
MKTFAAYVIIACLSSTALASSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEIRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 48 - DNA, 801 bp, Artificial Sequence
  hybrid
atgaaaacattagccgcatatttagttctagttttttatgcaagcaccgcgctctcagagtcaattacagaaaatttggcgtggaataaagaattttctagtgaatccgtacatggcgtttttgtactttgtaaaagtagtagcaattcctgtactacaaataatgcggcacgtgcatctacagcctatattccagcatcaacattcaaaattcctaatgctctaataggtcttgaaaccggcgccataaaagatgaacggcaggttttcaaatgggacggaaagccaagagccatgaagcaatgggaaagagacttaaagctaaggggcgctatacaggtttctgctgttccggtatttcaacaaattgccagagaagttggcgaaataagaatgcaaaaataccttaacctgttttcatacggcaacgccaatatagggggaggcattgacaaattctggctagaaggtcagcttagaatctcagcattcaatcaagttaaatttttagagtcgctctacctgaataaattgtcagcatcaaaagaaaaccaactaatagtaaaagaggcaatagttacagaagcaactccagaatatatagttcattcaaaaactggtttttctggtgttggcacagaatcaagtcctggtgtcgcttggtgggttggttgggtagagaaaggaactgaggtttacttttttgcttttaacatggacatagacaatgagagtaaattgccgtcaagaaaatccattcccaccaaaatcatggcaagtgagggcatcattggtggctaa

SEQ ID NO: 49 - protein, 266 aa, Artificial Sequence
  hybrid
MKTLAAYLVLVFYASTALSESITENLAWNKEFSSESVHGVFVLCKSSSNSCTTNNAARASTAYIPASTFKIPNALIGLETGAIKDERQVFKWDGKPRAMKQWERDLKLRGAIQVSAVPVFQQIAREVGEIRMQKYLNLFSYGNANIGGGIDKFWLEGQLRISAFNQVKFLESLYLNKLSASKENQLIVKEAIVTEATPEYIVHSKTGFSGVGTESSPGVAWWVGWVEKGTEVYFFAFNMDIDNESKLPSRKSIPTKIMASEGIIGG

SEQ ID NO: 50 - DNA, 801 bp, Artificial Sequence
  hybrid
atgaaaacatttgccgcatatgtaattatcgcgtgtctttcgagtacggcattagctggttcaattacagaaaatacgtcttggaacaaagagttctctgccgaagccgtcaatggtgtcttcgtgctttgtaaaagtagcagtaaatcctgcgctaccaatgacttagctcgtgcatcaaaggaatatcttccagcatcaacatttaagatccccaacgcaattatcggcctagaaactggtgtcataaagaatgagcatcaggttttcaaatgggacggaaagccaagagccatgaagcaatgggaaagagacttgaccttaagaggggcaatacaagtttcagctgttcccgtatttcaacaaatcgccagagaagttggcgaagtaagaatgcagaaatatcttaaaaaattttcatatggtaaccagaatatcagtggtggcattgacaaattctggttggaaggtcagcttagaatttccgcagttaatcaagtggagtttctagagtctctatttttaaataaattgtcagcatcaaaagaaaatcagctaatagtaaaagaggctttggtaacggaggctgcgcctgaatatcttgtgcattcaaaaactggtttttctggtgtgggaactgagtcaaatcctggtgtcgcatggtgggttggttgggttgagaagggagcagaggtttactttttcgcatttaacatggatatagacaacgaaaataagttgccgctaagaaaatccattcccaccaaaatcatggcaagtgagggcatcattggtggctaa

SEQ ID NO: 51 - protein, 266 aa, Artificial Sequence
  hybrid
MKTFAAYVIIACLSSTALAGSITENTSWNKEFSAEAVNGVFVLCKSSSKSCATNDLARASKEYLPASTFKIPNAIIGLETGVIKNEHQVFKWDGKPRAMKQWERDLTLRGAIQVSAVPVFQQIAREVGEVRMQKYLKKFSYGNQNISGGIDKFWLEGQLRISAVNQVEFLESLFLNKLSASKENQLIVKEALVTEAAPEYLVHSKTGFSGVGTESNPGVAWWVGWVEKGAEVYFFAFNMDIDNENKLPLRKSIPTKIMASEGIIGG

SEQ ID NO: 52 - DNA, 1704 bp, Artificial Sequence
  hybrid
atgatgtctgttgctactgttgaaccaccaaagccagaattgtctccaccacaaaaccaaaacgctccatcttctcacgaaactgatcacattttcagatctaagttgccagatattactatttctaacgatttgccattgcacgcttactgtttcgaaaacttgtctgatttctctgatagaccatgtttgatttctggttctactggtaagactttcacttacgctgatactcacttgatttcttctaagattgctgctggtttgtctaacttgggtattttgaagggtgatgttgttatgattttgttgcaaaactctgctgatttcgttttctctttcttggctatttctatgattggtgctgttgctactactgcttctccattctacactgctccagaaattttcaagcaattcactgtttctaaggaaaagttggttattactcaagctatgtacgttgataagttgagaaaccacgatggtgctaagttgggtgaagatttcattgttattactattgatgatccaccagaaaactgtttgcacttcaacgttttggttgaagcttctgaatctgaaatgccaactgtttctattttgccagatgatccagttgctttgccattctcttctggtactactggtttgccaaagggtgttattttgactcacaagtctttgattacttctgttgctcaacaagttgatggtgaaattccaaacttgtacttgaagcaagatgatgttgttttgtgtgttttgccattgttccacattttctctttgaactctgttttgttgtgttctttgagagctggttctgctgttttgttgatgcaaaagttcgaaattggttctttgttggaattgattcaaagacacagagtttctgttgctatggttgttccaccattggttttggctttggctaagaacccaatggttgctgatttcgatttgtcttctattagattggttttgtctggtgctgctccattgggtaaggaattggaagaagctttgagaaacagaatgccacaagctgttttgggtcaaggttacggtatgactgaagctggtccagttttgtctatgtgtttggctttcgctaagcaaccattccaaactaagtctggttcttgtggtactgttgttagaaacgctgaattgaaggttgttgatccagaaactggtagatctttgggttacaaccaaccaggtgaaatttgtattagaggtcaacaaattatgaagggttacttgaacgatgaagctgctactgcttctactattgattctgaaggttggttgcacactggtgatgttggttacgttgatgatgatgatgaaattttcattgttgatagagttaaggaaattattaagttcaagggtttccaagttccaccagctgaattggaagctttgttggttaaccacccatctattgctgatgctgctgttgttccacaaaaggatgaagttgctggtgaagttccagttgctttcgttgttagatctgatgatttggatttgtctgaagaagctgttaaggaatacattgctaagcaagttgttttctacaagaagttgcacaaggttttcttcgttcactctattccaaagtctgcttctggtaagattttgagaaaggatttgagagctaagttggctactgctactactcaaactccataa

SEQ ID NO: 53 - DNA, 1518 bp, Artificial Sequence
  hybrid
atggatttgttgttgttggaaaaggctttgatgggtttgttcttggctgctgttgttgctattgctgtttctactttgagaggtaagaagttgaagttgccaccaggtccaattccagttccagttttcggtaactggttgcaagttggtgatgatttgaaccaaagaaacttggttgattacgctaagaagttcggtgatttgttcatgttgagaatgggtcaaagaaacttggttgttgtttcttctccagaattggctaaggaagttttgcacactcaaggtgttgaattcggttctagaactagaaacgttgttttcgatattttcactggtaagggtcaagatatggttttcactgtttacggtgaacactggagaaagatgagaagaattatgactgttccattcttcactaacaaggttgttcaacaatacagacacggttgggaatctgaagctgctgctgttgttgaagatgttaagaagaacccagatgctgctactactggtactgttatgagaagaagattgcaattgatgatgtacaacaacatgtacagaattatgttcgatagaagattcgaatctgatgaagatccaatgttcttgagattgaaagctttgaacggtgaaagatctagattggctcaatctttcgaatacaactacggtgatttcattccaattttgagaccattcttgaagggttacttgaagatttgtaaggaagttaaggaaactagattgaagttgttcaaggattacttcgttgatgaaagaaagaagttggaatctactaagtctgctgacaacaactctttgaagtgtgctattgatcacattattgaagctcaaagaaagggtgaaattaacgaagataacgttttgtacattgttgaaaacattaacgttgctgctattgaaactactttgtggtctattgaatggggtattgctgaattggttaaccacccagaaattcaacaaaagttgagagatgaaattgatagagttttgggtgctggtcaccaagttactgaaccagatattcaaaagttgccatacttgcaagctgttgttaaggaaactttgagattcagaatggctattccattgttggttccacacatgaacttgcacgatgctaagttgggtggttacgatattccagctgaatctaagattttggttaacgcttggtggttggctaacaacccagctcactggaacaagccagaagaattcagaccagaaagattcttggaagaagaatctaaggttgaagctaacggtaacgatttcaaatacattccattcggtgttggtagaagatcttgtccaggtattattttggctttgccaattttgggtattgctatgggtagattggttcaaaacttcgaattgttgccaccaccaggtcaatctcaaattgatacttctgaaaagggtggtcaattctctttgcacattttgaagcactctactattgtttgtaagccaagatctttgtaa

SEQ ID NO: 54 - DNA, 705 bp, Artificial Sequence
  hybrid
atggctttcccatctgttacttctgttactgttgaaaacgttactttcccaccaactgttaagccaccatgttctccaaacactttcttcttggctggtgctggtgttagaggtttgcaaattcaccacgctttcgttaagttcactgctatttgtatttacttgcaatacgatgctttgtctttcttgtctgttaagtggaagactaagtctactcaccaattgactgaatctgatcaattcttctctgatattgttactggtccattcgaaaagttcatgcaagttactatgattttgccattgactggtcaacaatactctgaaaaggttgctgaaaactgtgttgctatttggagatctttgggtatttacactgattctgaagctgaagctattgataagttcttgtctgttttcaaggatttgactttcccaccaggtgcttctattttgttcactacttctccagatggttctttgactatttctttctctaaggatggtatgattccagaagctgctaacattgttttggaaaacgaaaagttggctcaagctgttattgaatctgttattggtaagaacggtgtttctccagctactaagcaatctttggcttctagattgtctgatttgatgaaccacttcgatgaaaaggctactactgatgctgaaccaaacttgtctaagaacggtttgtaa

SEQ ID NO: 55 - DNA, 1170 bp, Artificial Sequence
  hybrid
atggttactgttgaagaagttagaaaggctcaaagagctgaaggtccagctactgttatggctattggtactgctactccaccaaactgtgttgatcaatctacttacccagattactacttcagaattactaactctgaacacaagactgaattgaaggaaaagttccagagaatgtgtgaaaagtctatgattaagaagagatacatgcacttgactgaagaaattttgaaggaaaacccaaacatgtgtgaatacatggctccatctttggatgctagacaagatattgttgttgttgaagttccaaagttgggtaaggaagctgctcaaaaggctattaaggaatggggtcaaccaaagtctaagattactcacttggttttctgtactacttctggtgttgatatgccaggttgtgattaccaattgactaagttgttgggtttgagaccatctgttaagagattgatgatgtaccaacaaggttgtttcgctggtggtactgttttgagattggctaaggatttggctgaaaacaacaagggtgctagagttttggttgtttgttctgaaattactgctgttactttcagaggtccaaacgatactcacttggattctttggttggtcaagctttgttcggtgatggtgctgctgctattattattggttctgatccaattccaggtgttgaaagaccattgttcgaattggtttctgctgctcaaactattttgccagattctgaaggtgctattgatggtcacttgagagaagttggtttgactttccacttgttgaaggatgttccaggtttgatttctaagaacattgaaaagtctttggaagaagctttcaagccattgggtatttctgattggaactctttgttctggattgctcacccaggtggtccagctattttggatcaagttgaaattaagttgggtttgaagccagaaaagttgagagctactagacacgttttgtctgaatacggtaacatgtcttctgcttgtgttttgttcattttggatgaaatgagaagaaagtctaaggaagatggtttgaagactactggtgaaggtattgaatggggtgttttgttcggtttcggtccaggtttgactgttgaaactgttgttttgcactctgttgctacttaa

SEQ ID NO: 56 - DNA, 1131 bp, Artificial Sequence
  hybrid
atggctccaactgctaagactttgacttacttggctcaagaaaagactttggaatcttctttcgttagagatgaagaagaaagaccaaaggttgcttacaacgaattctctgatgaaattccagttatttctttggctggtattgatgaagttgatggtagaagaagagaaatttgtgaaaagattgttgaagcttgtgaaaactggggtattttccaagttgttgatcacggtgttgatgctgatttgattgctgatatgactagattggctagagaattcttcgctttgccagctgaagataagttgagatacgatatgtctggtggtaagaagggtggtttcattgtttcttctcacttgcaaggtgaagctgttcaagattggagagaaattgttacttacttctcttacccagttaaggctagagattacggtagatggccagaaaagccagctggttggtgtgctgttgttgaaagatactctgaaagattgatgggtttgtcttgtaacttgatgggtgttttgtctgaagctatgggtttggaaactgaagctttggctaaggcttgtgttgatatggatcaaaaggttgttgttaacttctacccaagatgtccacaaccagatttgactttgggtttgaagagacacactgatccaggtactattactttgttgttgcaagatttggttggtggtttgcaagctactagagatggtggtaagaactggattactgttcaaccaatttctggtgctttcgttgttaacttgggtgatcacggtcacttcatgtctaacggtagattcaagaacgctgatcaccaagctgttgttaacggtgaatcttctagattgtctattgctactttccaaaacccagctccagatgctagagtttggccattggctgttagagaaggtgaagaaccaattttggaagaaccaattactttcactgaaatgtacagaagaaagatggaaagagatttggatttggctaagagaaagaagcaagctaaggatcaattgatgcaacaacaattgcaattgcaacaacaacaagctgttgctgctgctccaatgccaactgctactaagccattgaacgaaattttggcttaa

SEQ ID NO: 57 - DNA, 1011 bp, Artificial Sequence
  hybrid
atgggtgttgaaagagttcaagatattgcttctactatttctgaagatactattccagctgaatacattagatctgaaaacgaacaaccaggtattactactgttccaaacactgttttggaatgtccaactattgatttctctgatccagatgaagaaaagttgttgaagcaaattttcgaagcttctattgattggggtatgtaccaaattgttaaccacgatatttctaacgaagctatttctaagttgcaagctgttggtaaggaattcttcgaagaattgccacaagaagaaaaggaagtttacgctaaggatccaaactctaagtctgttgaaggttacggtactttcttgcaaaaggaatttgaaggtaagaagggttgggttgatcacttgttccacaagatttggccaccatctgctattaactacagattctggccaaagaacccaccatcttacagagaagctaacgaagaatacgctaagtggttgagaaaggttgttgatgagttgttcaagttgttgtctttgggtttgggtttggaagctcaagaaatgaagaaggctgctggtgctgatgattttgtttacttgttgaagattaactactacccaccatgtccaagaccagatttggctttgggtgttgttgctcacactgatatgtcttacattactttgttggttccaaacgaagttcaaggtttgcaagttttcaaggatggtcactggtacgatgttaactacattccaaacgctattattgttcacattggtgatcaagttgaaattttgtctaacggtaagtacaagtctgtttaccacagaactactgttaacaagtacaagactagaatgtcttggccagttttcttggaaccatcttctgaacacgaagttggtccaattccaaagttgattaacgataaggaaaacccaccagagttcaagactaagaagtacaaggattacgtttactgtaagttgaacaagttgccacaataa

SEQ ID NO: 58 - DNA, 2148 bp, Artificial Sequence
  hybrid
atggaaactgttactaagaacggttaccaaaacggtaacggttggaatttgtgtatgaagaaggaagatccattgaactggggtgttgctgctgaagctttgactggttctcacttggatgaagttaagagaatggttgctgaatacagaaagccagttgttaagttggaaggtgaaactttgactatttctcaagttgctgctatttctgctagagatgattctggtgttaaggttgaattgtctgaagaagctagagctggtgttaaggcttcttctgattgggttatggattctatgaacaagggtactgattcttacggtgttactactggtttcggtgctacttctcacagaagaactaagcaaggtggtgctttgcaaaaggaattgattagattcttgaacgctggtattttcggtaacggtactgaaacttgtcacactttgccacactctgctactagagctgctatgttggttagaattaacactttgttgcaaggttactctggtattagattcgaaattttggaagctattactaagttcttgaaccacaacattactccatgtttgccattgagaggtactattactgcttctggtgatttggttccattgtcttacattgctggtttgttgactggtagaccaaactctaaggctgttggtccaactggtgttactttgtctccagaagaagctttcaagttggctggtgttgaaggtggtttcttcgaattgcaaccaaaggaaggtttggctttggttaacggtactgctgttggttctggtatggcttctatggttttgttcgaagctaacattttggctgttttggctgaagttatgtctgctattttcgctgaagttatgcaaggtaagccagaattcactgatcacttgactcacaagttgaagcaccacccaggtcaaattgaagctgctgctattatggaacacattttggatggttctgcttacgttaaggctgctcaaaagttgcacgaaatggatccattgcaaaagccaaagcaagatagatacgctttgagaacttctccacaatggttgggtccacaaattgaagttattagatcttctactaagatgattgaaagagaaattaactctgttaacgataacccattgattgatgtttctagaaacaaggctattcacggtggtaacttccaaggttctccaattggtgtttctatggataacactagattggctattgctgctattggtaagttgatgttcgctcaattctctgaattggttaacgatttctacaacaacggtttgccatctaacttgtctggtggtagaaacccatctttggattacggtttcaagggtgctgaaattgctatggcttcttactgttctgaattgcaattcttggctaacccagttactaaccacgttcaatctgctgaacaacacaaccaagatgttaactctttgggtttgatttcttctagaaagacttctgaagctgttgaaattttgaagttgatgtctactactttcttggttggtttgtgtcaagctattgatttgagacacttggaagaaaacttgaagtctactgttaagaacactgtttctcaagttgctaagagagttttgactatgggtgttaacggtgaattgcacccatctagattctgtgaaaaggatttgttgagagttgttgatagagaatacattttcgcttacattgatgatccatgttctgctacttacccattgatgcaaaagttgagagaaactttggttgaacacgctttgaacaacggtgaaaacgaaaagaacttctctacttctgttttccaaaagattgctgctttcgaagatgaattgaaggctttgttgccaaaggaagttgaaactgctagagctgctttggaatctggtaacccagctattccaaacagaattaaggaatgtagatcttacccattgtacaagttcgttagagaagaattgggtactgaatacttgactggtgaaaaggttagatctccaggtgaagaattcgaaaaggttttcactgctatgtctaagggtgaaattattgatccattgttggaatgtttggaatcttggaacggtgctccattgccaatttgttaa

SEQ ID NO: 59 - DNA, 22 bp, Artificial Sequence
  primer
ctgtgctgtctgcgctgcattc

SEQ ID NO: 60 - DNA, 23 bp, Artificial Sequence
  primer
ttacttgtgagattgtggatcac

SEQ ID NO: 61 - DNA, 21 bp, Artificial Sequence
  primer
atggctttcccatctgttact

SEQ ID NO: 62 - DNA, 21 bp, Artificial Sequence
  primer
atggctccaactgctaagact

SEQ ID NO: 63 - DNA, 23 bp, Artificial Sequence
  primer
ttaagccaaaatttccttcaatg

SEQ ID NO: 64 - DNA, 23 bp, Artificial Sequence
  primer
ttaacaaattggcaatggagaac

SEQ ID NO: 65 - DNA, 24 bp, Artificial Sequence
  primer
atggaaactgttactaagaacggt

SEQ ID NO: 66 - DNA, 22 bp, Artificial Sequence
  primer
ttaagtagcaacagagtgcaaa

SEQ ID NO: 67 - DNA, 25 bp, Artificial Sequence
  primer
atggttactgttgaagaatacagaa

SEQ ID NO: 68 - DNA, 24 bp, Artificial Sequence
  primer
ttagaaagatcttggcttagcaac

SEQ ID NO: 69 - DNA, 22 bp, Artificial Sequence
  primer
atggatttgttgttgttggaaa

SEQ ID NO: 70 - DNA, 22 bp, Artificial Sequence
  primer
ttattgtggcaacttgttcaac

SEQ ID NO: 71 - DNA, 24 bp, Artificial Sequence
  primer
atgaagactattcaaggtcaatct

SEQ ID NO: 72 - DNA, 21 bp, Artificial Sequence
  primer
ttatggagtttgagtagcagc

SEQ ID NO: 73 - DNA, 23 bp, Artificial Sequence
  primer
atgattactttggctccatcttt

SEQ ID NO: 74 - DNA, 20 bp, Artificial Sequence
  primer
gcgcatgtgtccgatctttg

SEQ ID NO: 75 - DNA, 21 bp, Artificial Sequence
  primer
atggctttcccatctgttact

SEQ ID NO: 76 - DNA, 21 bp, Artificial Sequence
  primer
atggctccaactgctaagact

SEQ ID NO: 77 - DNA, 24 bp, Artificial Sequence
  primer
atggaaactgttactaagaacggt

SEQ ID NO: 78 - DNA, 22 bp, Artificial Sequence
  primer
ttaagtagcaacagagtgcaaa

SEQ ID NO: 79 - DNA, 22 bp, Artificial Sequence
  primer
atggatttgttgttgttggaaa

SEQ ID NO: 80 - DNA, 22 bp, Artificial Sequence
  primer
ttattgtggcaacttgttcaac

SEQ ID NO: 81 - DNA, 24 bp, Artificial Sequence
  primer
atgaagactattcaaggtcaatct

SEQ ID NO: 82 - DNA, 21 bp, Artificial Sequence
  primer
ttatggagtttgagtagcagc

SEQ ID NO: 83 - DNA, 20 bp, Artificial Sequence
  primer
caccagcgctctagatacat

SEQ ID NO: 84 - DNA, 22 bp, Artificial Sequence
  primer
tgtgttaccgatatcggcgaat

SEQ ID NO: 85 - DNA, 21 bp, Artificial Sequence
  primer
tcattaccgcttccaagtcta

SEQ ID NO: 86 - DNA, 22 bp, Artificial Sequence
  primer
acgcagaattttcgagttatta

SEQ ID NO: 87 - DNA, 23 bp, Artificial Sequence
  primer
agaagtgtcaacaacgtatctac

SEQ ID NO: 88 - DNA, 24 bp, Artificial Sequence
  primer
ggtggtaatgccatgtaatatgat

SEQ ID NO: 89 - DNA, 25 bp, Artificial Sequence
  primer
ttcggtttgtattacttcttattca

SEQ ID NO: 90 - DNA, 22 bp, Artificial Sequence
  primer
tacatgcgtacacgcgtctgta

SEQ ID NO: 91 - DNA, 23 bp, Artificial Sequence
  primer
ccaattggttcaagtctccaaat

SEQ ID NO: 92 - DNA, 22 bp, Artificial Sequence
  primer
acgcagaattttcgagttatta

SEQ ID NO: 93 - DNA, 22 bp, Artificial Sequence
  primer
ctactgcctagcatcttgctaa

SEQ ID NO: 94 - DNA, 20 bp, Artificial Sequence
  primer
aatagggacctagacttcag

SEQ ID NO: 95 - DNA, 20 bp, Artificial Sequence
  primer
acgcacactactctctaatg

SEQ ID NO: 96 - DNA, 24 bp, Artificial Sequence
  primer
cccttacaagaacattcacgaaat

SEQ ID NO: 97 - DNA, 21 bp, Artificial Sequence
  primer
agacggtaggtattgattgta

SEQ ID NO: 98 - DNA, 22 bp, Artificial Sequence
  primer
cgacctcatgctatacctgaga

SEQ ID NO: 99 - DNA, 20 bp, Artificial Sequence
  primer
ctacttcgagcggaggcatc

SEQ ID NO: 100 - DNA, 20 bp, Artificial Sequence
  primer
tcattgccttctccactctc

(19) United States
(12) Patent Application Publication
(10) Pub. No.: US 2014/0200145 A1
(43) Pub. Date: Jul. 17, 2014

(54) METHOD OF METABOLIC EVOLUTION

(71) Applicants: Rudy Pandjaitan, Maisons Alfort (FR); Sarra Sebai, Paris (FR); Alejándro Luque, Paris (FR)

(72) Inventors: Rudy Pandjaitan, Maisons Alfort (FR); Sarra Sebai, Paris (FR); Alejándro Luque, Paris (FR)

(73) Assignee: Eviagenics S.A., Paris (FR)

(21) Appl. No.: 14/125,900

(22) PCT Filed: Jun. 20, 2012
(86) PCT No.: PCT/EP2012/061877

§ 371 Date: Feb. 18, 2014

Related U.S. Application Data
(60) Provisional application No. 61/499,535, filed on Jun. 21, 2011.

(51) Int. Cl. C12N 15/10

(52) U.S. Cl. CPC C12N 15/1058
USPC 506/1; 506/14; 435/252.33; 435/252.31; 435/254.21; 435/348; 435/367; 435/366; 435/254.22; 435/254.23; 435/254.2

(57) ABSTRACT

The invention relates to a method for metabolic evolution of a variant of a natural small aromatic molecule product of a metabolic pathway, by somatic in vivo assembly and recombination of said metabolic pathway employing a gene mosaic of at least one gene A, which comprises a) in a single step procedure (i) transforming a cell with at least one gene A having a sequence homology of less than 99.5% to another gene to be recombined that is an integral part of the cell genome or presented in the framework of a genetic construct, (ii) recombining said genes, (iii) generating a gene mosaic of the genes at an integration site of a target genome, wherein said at least one gene A has a single flanking target sequence either at the 5′ end or 3′ end anchoring to the 5′ or 3′ end of said integration site, (iv) recombining eventual further genes of said metabolic pathway, and b) selecting clones comprising said gene mosaic and said eventual further genes capable of expressing said variant, methods of preparing a library of cells producing variants of natural small aromatic molecule products of a metabolic pathway, the libraries so produced and used to prepare said variants.

The invention refers to methods for metabolic evolution of variants of a natural small aromatic molecule product of a metabolic pathway by somatic in vivo assembly and recombination of said metabolic pathway employing gene mosaics.

### BACKGROUND

One of the primary goals of protein design is to generate proteins with new or improved properties. The ability to confer a desired activity on a protein or enzyme has considerable practical application in the chemical and pharmaceutical industry. Directed protein evolution has emerged as a powerful technology platform in protein engineering, in which libraries of variants are searched experimentally for clones possessing the desired properties.

Directed protein evolution harnesses the power of natural selection to evolve proteins or nucleic acids with desirable properties not found in nature. Various techniques are used for generating protein mutants and variants and selecting desirable functions. Recombinant DNA technologies have allowed the transfer of single structural genes or genes for an entire pathway to a suitable surrogate host for rapid propagation and/or high-level protein production. Accumulated improvements in activity or other properties are usually obtained through iterations of mutation and screening. Applications of directed evolution are mainly found in academic and industrial laboratories to improve protein stability and enhance the activity or overall performance of enzymes and organisms or to alter enzyme substrate specificity and to design new activities. Most directed evolution projects seek to evolve properties that are useful to humans in an agricultural, medical or industrial context (biocatalysis).

The evolution of whole metabolic pathways is a particularly attractive concept, because most natural and novel compounds are produced by pathways rather than by single enzymes. Metabolic pathways engineering usually requires the coordinated manipulation of all enzymes in the pathway. The evolution of new metabolic pathways and the enhancement of bioprocessing usually is performed through a process of iterative cycles of recombination and screening or selection to evolve individual genes, whole plasmids, multigene clusters, or even whole genomes.

Shao et al (1) describe the assembly of large recombinant DNA encoding a whole biochemical pathway or genome in a single step via in vivo homologous recombination of two flanking (anchoring) regions at the 5′ and 3′ ends containing sequences of the 5′ or 3′ end of the adjacent fragment in Saccharomyces cerevisiae.

Elefanty et al. (2) describe gene targeting experiments to generate mutant mice, in which the lacZ reporter gene has been knocked in to the SCL locus. Reference is made to FIG. 1 showing the SCL-lacZ gene targeting strategy employing two anchoring sequences, i.e. one at each of the the 5′ and 3′ end.

U.S. Pat. No. 7,807,422B2 (3) discloses the production of flavonoids by recombinant microorganisms. A set of genes is introduced into a heterologous host cell, such that the expression of the genes results in the production of the enzymes.

Naesby et al. (4) describe the random assembly of biosynthetic pathways and production of diverse natural products or intermediates in yeast. Genes encoding enzymes of a seven step flavonoid pathway were individually cloned into yeast expression cassettes, which were then randomly combined on Yeast Artificial Chromosomes. Similarly, Trantas et al (5) were able to express heterologous genes coding for flavonoid and stilbene pathway enzymes in yeast plasmids.

Directed evolution can be performed in living cells, also called in vivo evolution, or may not involve cells at all (in vitro evolution). In vivo evolution has the advantage of selecting for properties in a cellular environment, which is useful when the evolved protein or nucleic acid is to be used in living organisms. In vivo homologous recombination in yeast has been widely used for gene cloning, plasmid construction and library creation.

Library diversity is obtained through mutagenesis or recombination. DNA shuffling allows the direct recombination of beneficial mutations from multiple genes. In DNA shuffling a population of DNA sequences are randomly fragmented and then reassembled into full-length hybrid sequences.

For the purpose of homologous recombination naturally occurring homologous genes are used as the source of starting diversity. Single-gene shuffling library members are typically more than 95% identical. The family-shuffling, however, allows block exchanges of sequences that are typically more than 60% identical. The functional sequence diversity comes from related parental sequences that have survived natural selection; thus, much larger numbers of mutations are tolerated in a given sequence without introducing deleterious effects on the structure or function.

The recombination of DNA fragments of different origin with up to 30% diversity is described in WO1990007576A1 (6). Hybrid genes are produced in vivo by intergeneric and/or interspecific recombination in mismatch repair deficient bacteria or in bacteria of which the mismatch repair (MMR) system is transitorily inactivated. Thereby those processes by which damaged DNA are repaired, are avoided, which would have an inhibitory effect on the recombination frequency between divergent sequences, i.e. homeologous recombination.

The diversity of libraries can be enhanced by taking advantage of the ability of haploid cells to efficiently mate leading to the formation of a diploid organism. In its vegetative life cycle S. cerevisiae cells have a haploid genome, i.e. every chromosome is present as a single copy. Under certain conditions the haploid cells can mate. By this way a diploid cell is formed. Diploid cells can form haploid cells again, especially when certain nutrients are missing. They then undergo a process called meiosis followed by sporulation to form four haploid spores. During meiosis the different chromosomes of the two parental genomes recombine. During meiotic recombination DNA fragments are exchanged resulting in recombined DNA material.

WO2005/075654A1 (7) discloses a system for generating recombinant DNA sequences in Saccharomyces cerevisiae, which is based on the sexual reproductive cycle of S. cerevisiae. Heterozygous diploid cells are grown under conditions which induce the processes of meiosis and spore formation. Meiosis is generally characterized by elevated frequencies of genetic recombination. Thus, the products of meiosis, which are haploid cells or spores, can contain recombinant DNA sequences due to recombination between the two diverged DNA sequences. By an iterative method recombinant haploid progeny is selected and mated to one another, the resulting diploids are sporulated again, and their progeny spores are subjected to appropriate selection conditions to identify new recombination events. This process is described in wild-type or mismatch repair defective S. cerevisiae cells. Therefore, the genes of interest, each flanked by two selection markers, are integrated into an identical locus of each of the two sister chromosomes of mismatch repair deficient diploid strains. DNA sequences are added to the 5′ or 3′ end of the new DNA fragment that are 100% identical to the flanking DNA sequences of the locus where the DNA has to be integrated. These flanking target sequences are about 400-450 nucleotides long. Then the cells are forced to initiate sporulation. During the sporulation the recombination process takes place. The resulting spores and recombinant sequences can be differentiated by selection for the appropriate flanking markers.

The ability of yeast to efficiently recombine homologous DNA sequences can also be exploited to increase the diversity of a library. When two genes that share 89.9% homology were mutated by PCR and transformed into wild type yeast, a chimeric library of 10e7 was created through in vivo homologous recombination, showing several cross-over points throughout the two genes (8).

Though efforts were made to improve metabolic pathways by engineering recombinant hosts to produce small molecules on an industrial scale, variants of such metabolic products have only been sporadically found, e.g. by incomplete pathways resulting in the accumulation of intermediates.

It is the object of the present invention to provide an improved method for producing variants of natural small aromatic molecules as products of a metabolic pathway.

The object is achieved by the provision of the embodiments of the present application.

### SUMMARY OF THE INVENTION

According to the invention there is provided a method for metabolic evolution of a variant of a natural small aromatic molecule product of a metabolic pathway, by somatic in vivo assembly and recombination of said metabolic pathway employing a gene mosaic of at least one gene A, which comprises
a) in a single step procedure
   (i) transforming a cell with at least one gene A having a sequence homology of less than 99.5% to another gene to be recombined that is an integral part of the cell genome or presented in the framework of a genetic construct,
   (ii) recombining said genes,
   (iii) generating a gene mosaic of the genes at an integration site of a target genome, wherein said at least one gene A has a single flanking target sequence either at the 5′ end or 3′ end anchoring to the 5″ or 3″end of said integration site,
   (iv) recombining eventual further genes of said metabolic pathway, and
b) selecting clones comprising said gene mosaic and said eventual further genes capable of expressing said variant.

It is specifically preferred that a selection marker is used in the gene mosaic and the clones are selected according to the presence of the selection marker. For example, the gene mosaic comprises a selection marker, e.g. where said gene A is linked to a selection marker. Alternatively, selection may also be made by the presence of any product resulting of recombinants, e.g. through determining the yield or functional characteristics. Specifically one or more different selection markers may be used to differentiate the type of gene mosaics.

Selection markers useful for the inventive method can be selected from the group consisting of any of the known nutrition auxotrophic markers, antibiotics resistance markers, fluorescent markers, knock-in markers, activator/binding domain markers and dominant recessive markers and colorimetric markers. Preferred markers can be temporally inactivated or functionally knocked out, and may be re-established to regain its marking property. Further preferred markers are traceable genes, wherein the marker is a function of either of the gene sequences A and/or the other gene(s), such as gene B, without separate sequences with a marker function, so that the expression of the gene mosaic can be directly determined through detection of the mosaic itself. In this case the gene mosaic is directly traceable.

According to a specific embodiment, said genes are comprised in a linear polynucleotide, a vector or a yeast artificial chromosome. Specifically, gene A and/or other genes to be recombined are in the form of linear polynucleotides, preferably of 300 to 20.000 bp. Specifically, there would be no need to construct or employ plasmids or megaplasmids. The gene(s) can thus be used as such, i.e. without carrier.

The genes used for recombination and integration can also be comprised in any genetic construct, e.g. to be used as vector for carrying said gene(s). Said genes can thus be comprised in a genetic construct, e.g. a linear polynucleotide, a vector or a yeast artificial chromosome. These preferably include linear polynucleotides, plasmids, PCR constructs, artificial chromosomes, like yeast artificial chromosomes, viral vectors or transposable elements.

According to a specific embodiment of the invention the integration site of the target genome is located on either of the genes, e.g. within a linear polynucleotide, a plasmid or chromosome, including artificial chromosomes.

Specifically, said another gene is part of the target genome, e.g. the genome of the cell. In a preferred embodiment said another gene is gene B being part of the genome of the cell.

According to an alternatively preferred embodiment, said another gene is a genetic construct separate from the target genome, such as a linear polynucleotide, and optionally integrated into the target genome in the course of the recombination.

According to a specific aspect of the method according to the invention, the cell is co-transformed with at least one gene A and at least one gene B, wherein said single flanking target sequence of gene A is anchoring to the 5′end of an integration site on said target genome, and wherein gene B is linked to a single flanking target sequence anchoring to the 3′ end of the integration site.

Specifically, the cell can be co-transformed with at least one gene A with a selection marker and at least one gene B, wherein said single flanking target sequence of gene A is anchoring to the 5′end of an integration site on said target genome, and wherein gene B is linked to a different selection marker and a single flanking target sequence anchoring to the 3′ end of the integration site, and wherein clones for the at least two selection markers are selected.

Specifically, the cell is co-transformed with at least two different genes A1 and A2 and optionally with at least two different genes B1 and B2.

According to a further aspect of the method according to the invention, at least one further gene C is co-transformed, which has a sequence hybridizing with a sequence of gene A and/or said another gene, e.g. gene B, to obtain assembly and eventual recombination of said further gene C to gene A and/or said another gene.

Specifically, the hybridizing sequence of said gene C has a sequence homology of less than 99.5% to said sequence, and preferably at least 30% sequence homology.

Specifically gene mosaics having at least one nucleotide exchange or cross-over within the genes are selected, i.e. mosaics with an intragenic cross-over, such as those comprising parts of gene A and parts of said another gene(s) combined, which is understood as a mixture of partial genes to obtain a recombined intragenic gene mosaic, such as genes suitable for the expression of products in a different way, e.g. having improved properties or at improved yields. Such intragenic gene mosaics can be produced by recombination and preferably also assembly of a series of genes, wherein one or more of the assembled genes have such intragenic gene mosaics.

According to a preferred embodiment, mosaics of at least three different genes A and/or B and/or C can be obtained.

Preferably, said gene A and/or said another gene is a non-coding sequence or a sequence coding for a polypeptide or part of a polypeptide having an activity.

Specifically, the inventive method employs genes A, B and/or C which are coding for part of a polypeptide having an activity. Accordingly, the genes, such as genes A and/or B and/or C, preferably all of them do not individually encode a biologically active polypeptide as such, but would encode only part of it, and may bring about a respective activity or modified activity upon gene assembly only.

Using the inventive method, multiple genes coding for polypeptides of a biochemical pathway can be assembled and recombined. According to a preferred embodiment, at least two genes of said metabolic pathway are recombined and assembled, for example, at least two genes coding for polypeptides of said metabolic pathway are recombined and assembled.

Specifically, said genes are linear polynucleotides, preferably of 300 to 20.000 bp.

According to a specific embodiment, gene mosaics of at least 3, preferably at least 9, up to 20.000 base pairs, preferably with at least 3 cross-over events per 700 bp are obtained. Gene mosaics are preferred, which comprise at least one intragenic mosaic, preferably with at least 3 cross-over events, preferably at least 4, 5, or 10 cross-over events per 700 base pairs, more preferably per 600 bp, per 500 bp or even below. Typically a high degree of cross-over events provides for a large diversity of recombined genes, which may be used to produce a library for selecting suitable library members. The degree of mosaics or cross-over events can be understood as a quality parameter of such a library.

The method according to the invention specifically provides for the selection of at least one clone having an intragenic gene mosaic. Specifically, at least one clone having a gene assembly and at least one intragenic gene mosaic is selected.

The genes which are modified according to the method of the invention can be any genes useful for enzymatic processing of source material to produce small organic molecules, e.g. for scientific or industrial purposes. These genes may encode, for example, variants of polypeptides, in whole or in part, including those partial sequences, which do not encode a polypeptide with biological activity, which polypeptides are specifically selected from the group consisting of enzymes, transcription factors, transport proteins, signal peptides, receptors, hormones, growth factors, but also may encode non polypeptide genes such as promoters, terminators and other regulation factors, that may improve expression of the products.

Thus, recombination and/or assembly of sequences for “metabolic evolution”, including “enzymatic evolution” or “enzymatic synthesizing” as understood herein, refers to sequence mosaics supporting the enzymatic processing of a metabolic pathway, such as by enzyme variants or by compounds improving expression or activity of enzymes, including cofactors, but also non-coding sequences.

If genes are modified, which encode an amino acid sequence as part of a polypeptide having a biological activity, also called “partial genes”, it may be preferred that an assembly of such partial genes has functional features, e.g. encodes a polypeptide having a biological activity. Preferably a number of different genes, e.g. different partial genes, at a size ranging from 3 bp to 20.000 bp, specifically at least 100 bp, preferably from 300 bp to 20.000 bp, specifically up to 10.000 bp, are recombined, which number of different genes of is at least 2, more specifically at least 3, 4, 5, 6, 7, 8, 9, or at least 10 to produce a recombined gene sequence that is encoding a recombinant polypeptide, e.g. having a biological activity, which is advantageously modulated, e.g. having an increased biological activity. The term “biological activity” as used in this regard specifically refers to an enzymatic activity, such as an activity that converts a particular substrate into a particular product. Preferred genes as diversified according to the invention are coding for multi-chain polypeptides.

The method according to the invention specifically refers to the natural small aromatic molecules which are selected from the group consisting of phenylpropanoids, flavonoids, flavanols, anthocyanines, lignins, cyanidins, chalcones, vanillin, and naturally occurring derivatives thereof, always including intermediates.

In particular, said variants are synthesized by recombinant enzyme variants.

In a specific embodiment enzyme variants are obtained by such gene mosaics, e.g. directly by recombination and eventual assembly of the gene mosaics, or as a consequence of such gene mosaic, e.g. through a sequence of enzymatic processes. An exemplary method refers to cinnamate-4-hydrolase (C4H) and C4H generated genes coding for enzymes having improved or new enzymologic properties.

4-coumaroyl CoA is a pivotal molecule in the polypropanoid metabolism and it is also the substrate for ligase 4CL, an important branching point for defining the synthesis of flavonoids and stilbenes. The key enzyme CHS synthesizes chalcones that are used as intermediates for the synthesis of both flavonoids and isoflavones.

According to a preferred embodiment, said variant is a phenylpropanoid with biological activity selected from the group of antibacterial, antioxidative, fragrant and flavourful activity, e.g. as determined by a functional assay.

A specifically preferred method employs recombination and assembly of enzymes and enzyme pathways, comprising at least 2 enzymes having biological activity, to obtain enzyme variants or pathway variants having respective gene mosaics, for processing biological source material or arrays to produce such variants of natural small aromatic molecules with new structure and function. Thereby it was the first time possible to synthesize new small molecules through enzymatic evolution.

Any recombination competent eukaryotic or prokaryotic host cell can be used for generating a gene mosaic by somatic in vivo recombination according to the present invention. According to a preferred embodiment of the invention, the cell is a repair deficient cell, e.g. a nucleic acid repair deficient cell, such as with DNA repair deficiency, i.e. a DNA repair deficient cell, or an MMR deficient cell.

Specifically, the cell is a eukaryotic cell, preferably a fungal, mammalian or plant cell, or prokaryotic cell.

Preferably the cell is an Aspergillus sp or a fungal cell, preferably, it can be selected from the group consisting of the genera Saccharomyces, Candida, Kluyveromyces, Hansenula, Schizosaccaromyces, Yarrowia, Pichia and Aspergillus.

Preferably haploid strains, such as haploid yeast strains are employed.

Alternatively, prokaryotes, such as E. coli, Bacillus, Streptomyces, or mammalian cells, like HeLa cells or Jurkat cells, or plant cells, like Arabidopsis, may be used.

According to a specific embodiment, the flanking target sequence is at least 5 bp, preferably at least 10 bp, more preferably at least 20 bp, 50 bp, 100 bp up to 5,000 bp length. Specifically the flanking target sequence is linked to said gene or is an integral, terminal part of said gene. It is preferred that said the flanking target sequence has homology in the range of 30% to 99.5%, preferably less than 95%, less than 90%, less than 80%, hybridising with the anchoring sequence of said integration site,

When at least two different flanking target sequences anchoring to the target integration site of the genome are used according to the invention, it is preferred that they do not recombine with each other, preferably they share less than 30% homology.

According to a particular embodiment of the invention there is provided a method of cell display of gene variants, comprising creating a variety of gene mosaics in cells using the method according to the invention, and displaying said variety on the surface of said cells to obtain a library of mosaics.

According to a specific aspect of the invention there is provided a library of cells producing variants of natural small aromatic molecule products of a metabolic pathway, comprising engineering recombinant cells by somatic in vivo assembly and recombination of said metabolic pathway employing a gene mosaic of at least one gene A, which comprises
a) in a single step procedure
   (i) transforming a cell with at least one gene A having a sequence homology of less than 99.5% to another gene to be recombined that is an integral part of the cell genome or presented in the framework of a genetic construct,
   (ii) recombining said genes,
   (iii) generating a gene mosaic of the genes at an integration site of a target genome, wherein said at least one gene A has a single flanking target sequence either at the 5′ end or 3′ end anchoring to the 5″ or 3″end of said integration site,
   (iv) recombining eventual further genes of said metabolic pathway, and
b) collecting clones comprising said gene mosaic and said eventual further genes to obtain a library capable of producing said variants.

According to another aspect of the invention there is provided a library obtainable by such a method, comprising at least 10E3 different clones producing said variants. containing at least 1%, more preferred at least 10%, more preferred at least 20%, more preferred at least 40%, more preferred at least 60%, more preferred at least 80%, even more preferred at least 90%, more preferably at least 95% functional ORF's.

It is well understood that the term “obtainable” also refers to products obtained by methods.

According to a specific embodiment, a library of cells comprising recombinant genes encoding a repertoire of metabolic pathways is provided, which is obtainable by a method according to the invention.

Specifically, the library is a library of cells comprising recombinant genes encoding a repertoire of synthesizing enzymes.

Specifically, a library of synthesizing recombinant enzymes is provided, which is obtainable from such a library obtainable by the method according to the invention.

The library obtainable by such preferred display specifically comprises a high percentage of gene mosaics within a functional open reading frame (ORF), preferably at least 80%.

A library according to the invention specifically may be in any suitable form, specifically a biological library comprising a variety of organisms containing the gene variants. The biological library according to the invention may be contained in and/or specifically expressed by a population of organisms to create a repertoire of organisms, wherein individual organisms include at least one library member.

According to a specific aspect of the invention there is further provided an organism that comprises a gene variant from such a library, e.g. an organism selected from a repertoire of organisms. The organism as provided according to the invention may be used to express a gene expression product in a suitable expression system, e.g. as a production host cell.

The method according to the invention preferably further provides for selecting a variant of a natural small aromatic molecule from a library according to the invention e.g. through functional assays.

It is preferred to further characterize the variant, e.g. through determining the structure and function of said variant.

In a specific embodiment the method further comprises producing said variant in a recombinant host.

In another specific embodiment the method further comprises synthetically producing said variant.

### FIGURES

FIG. 1: Non-meiotic in vivo recombination

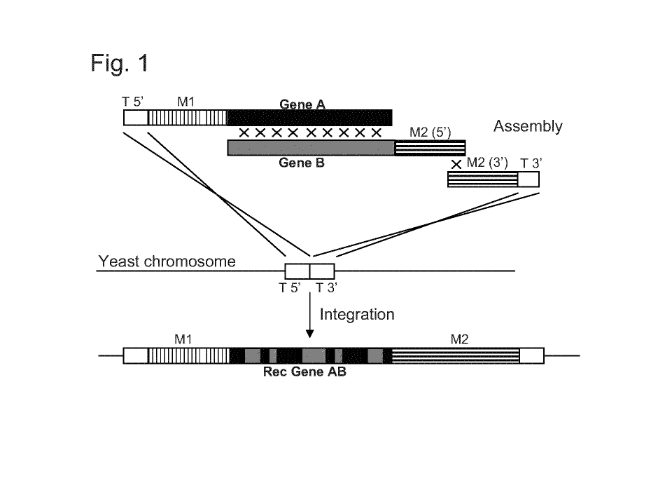

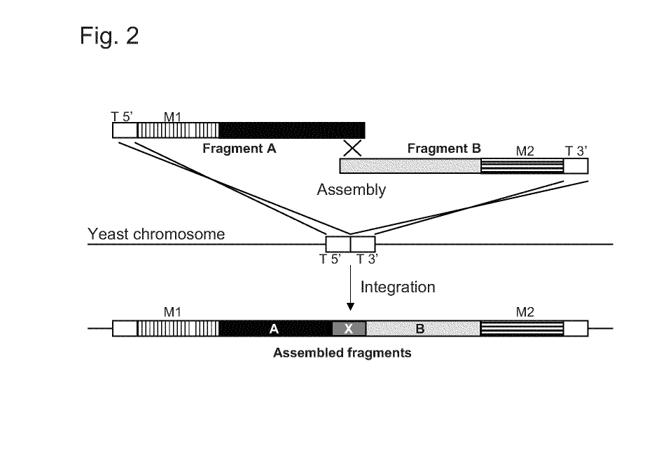

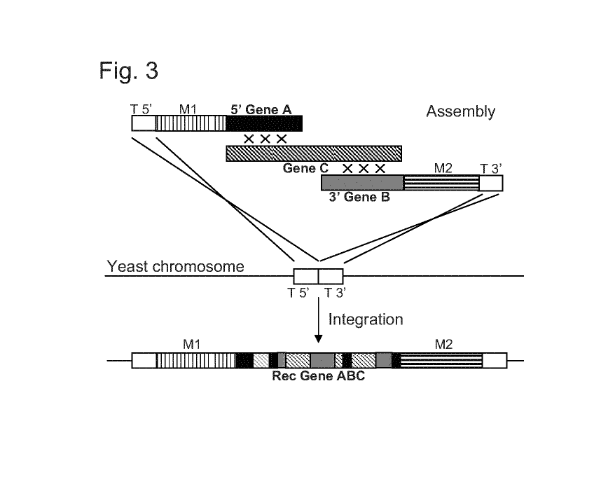

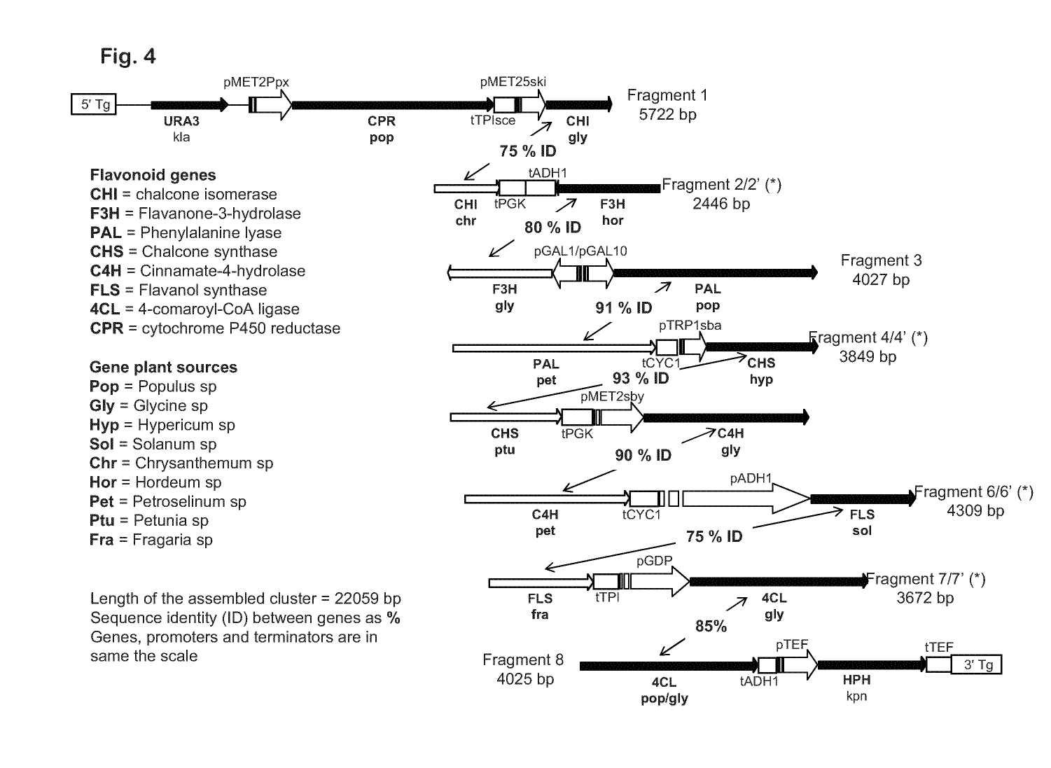

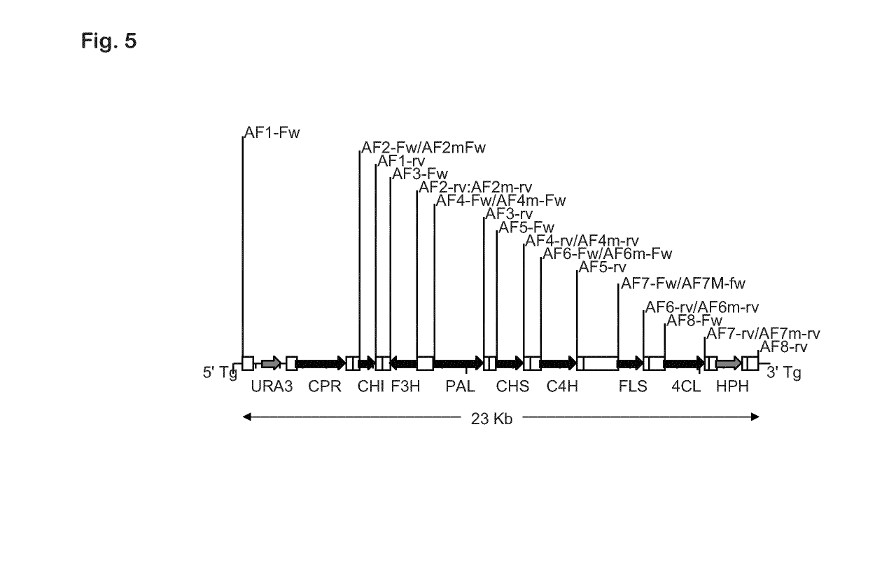

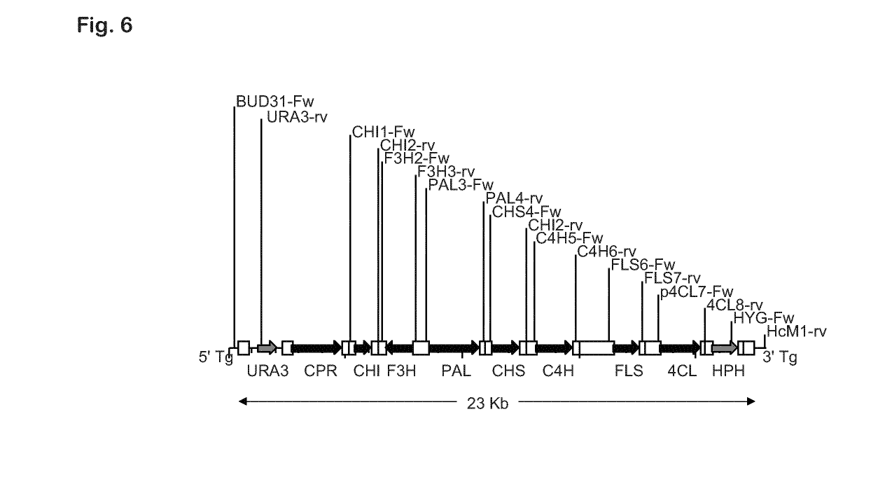

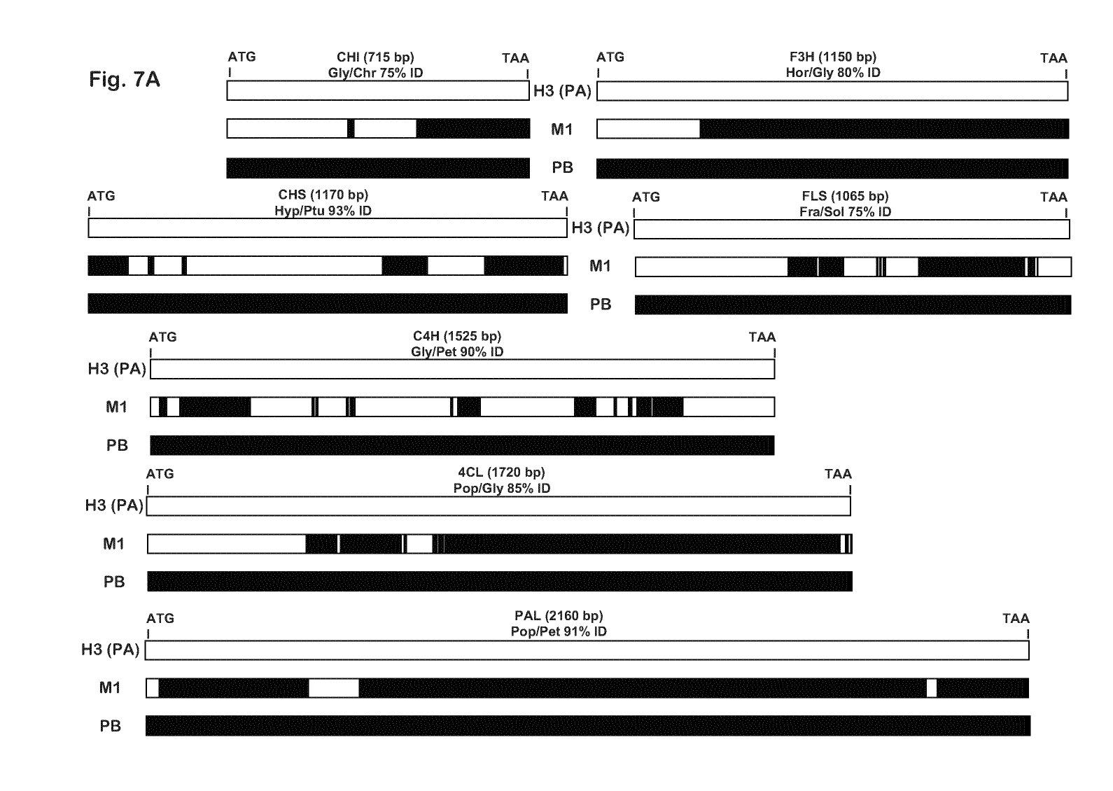

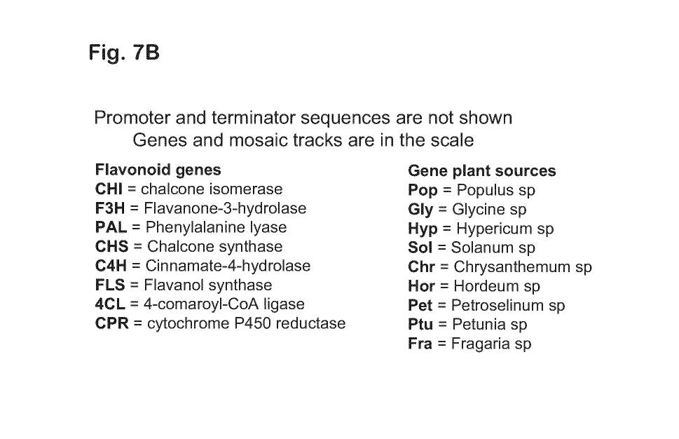

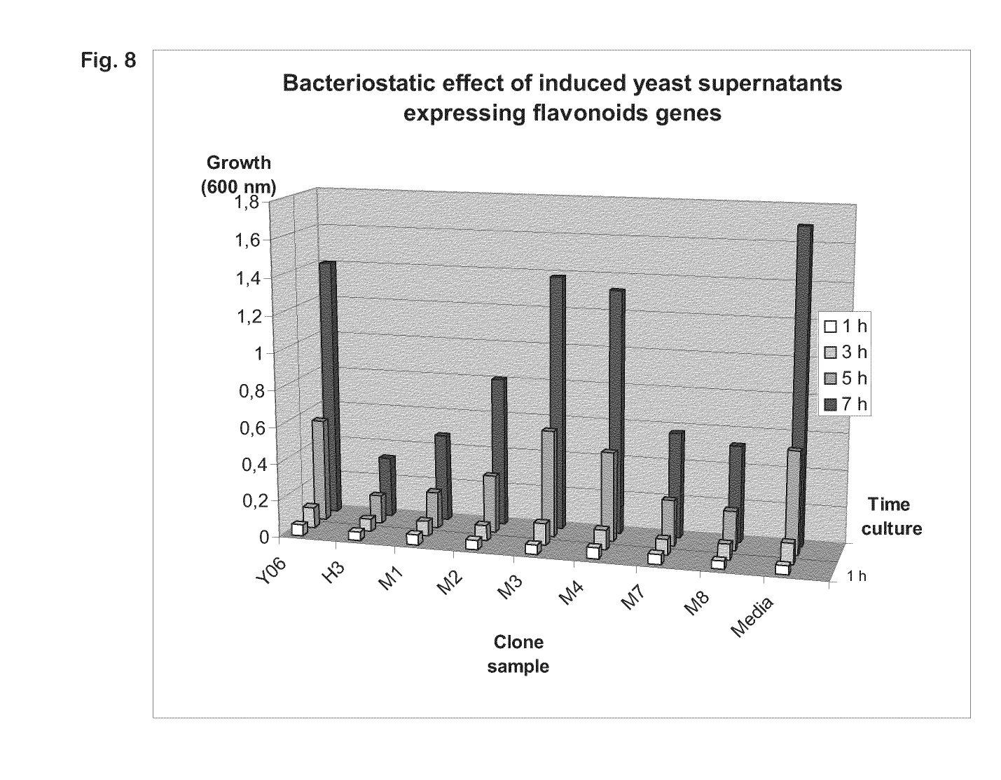

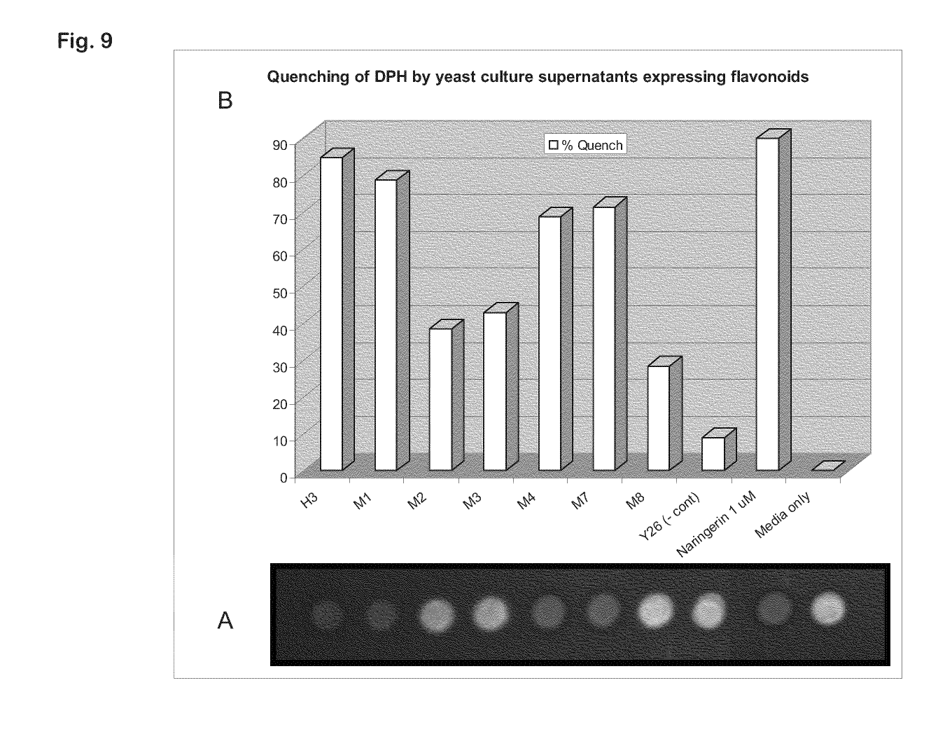

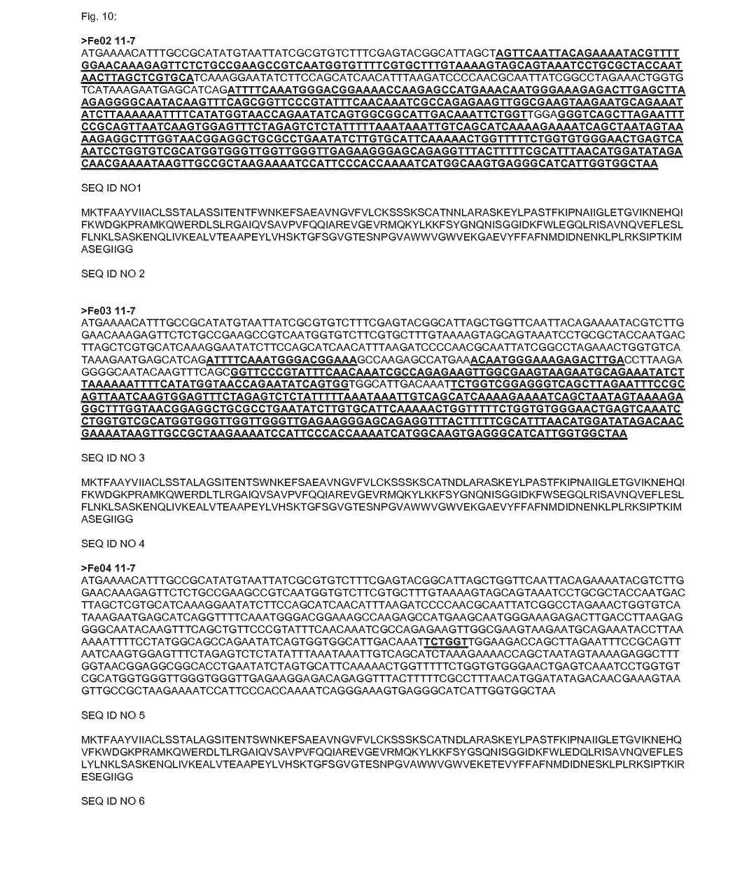

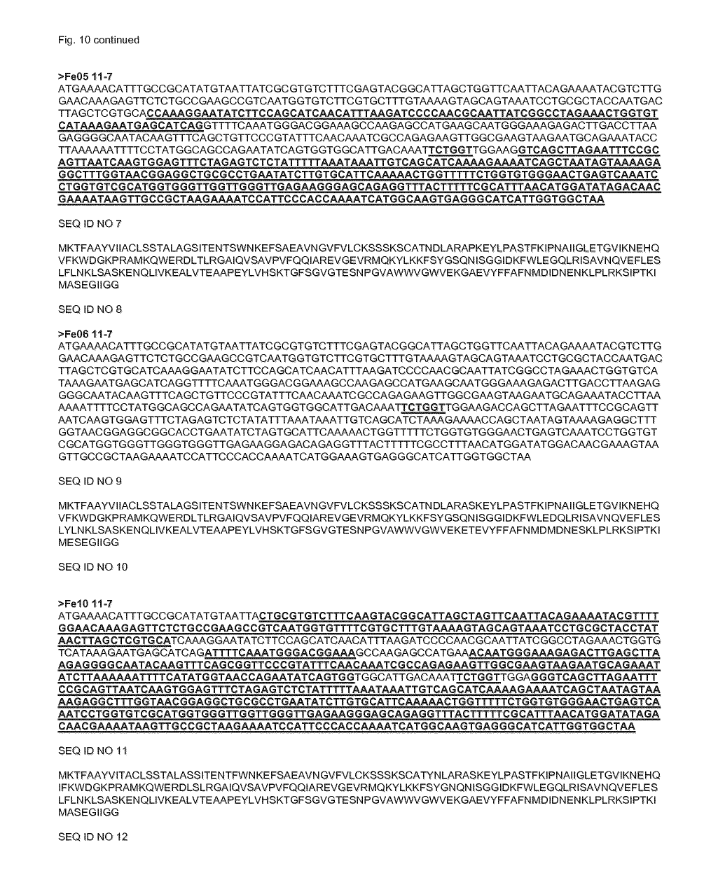

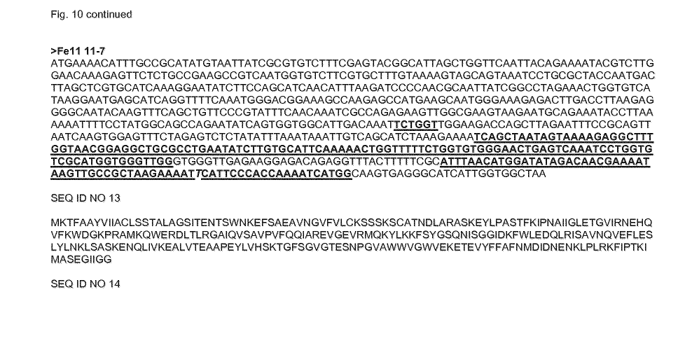

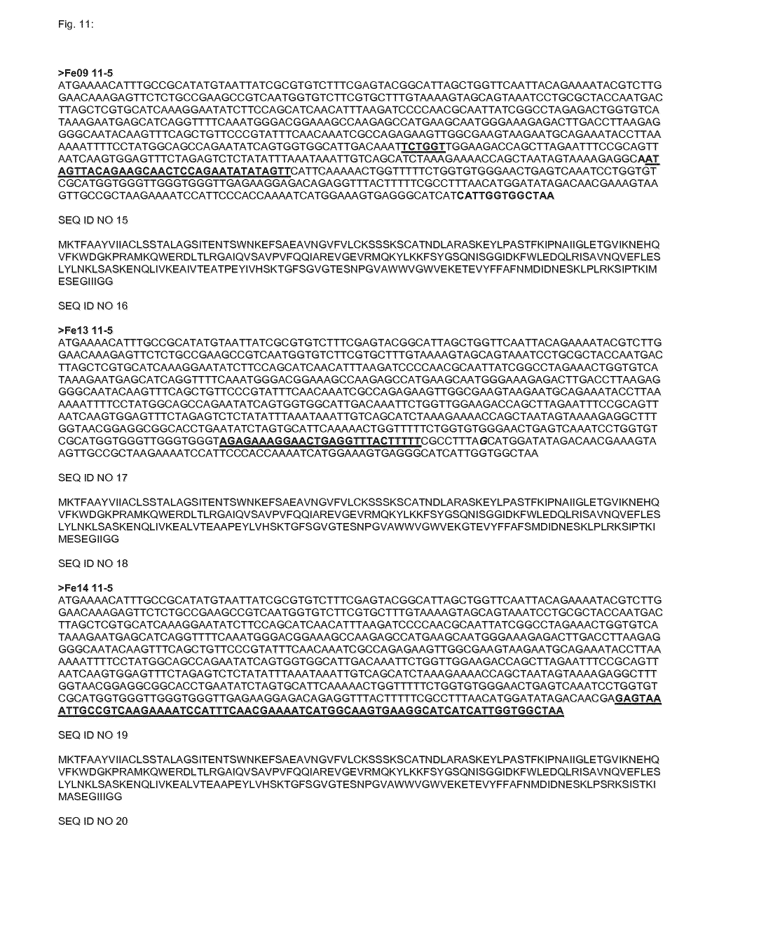

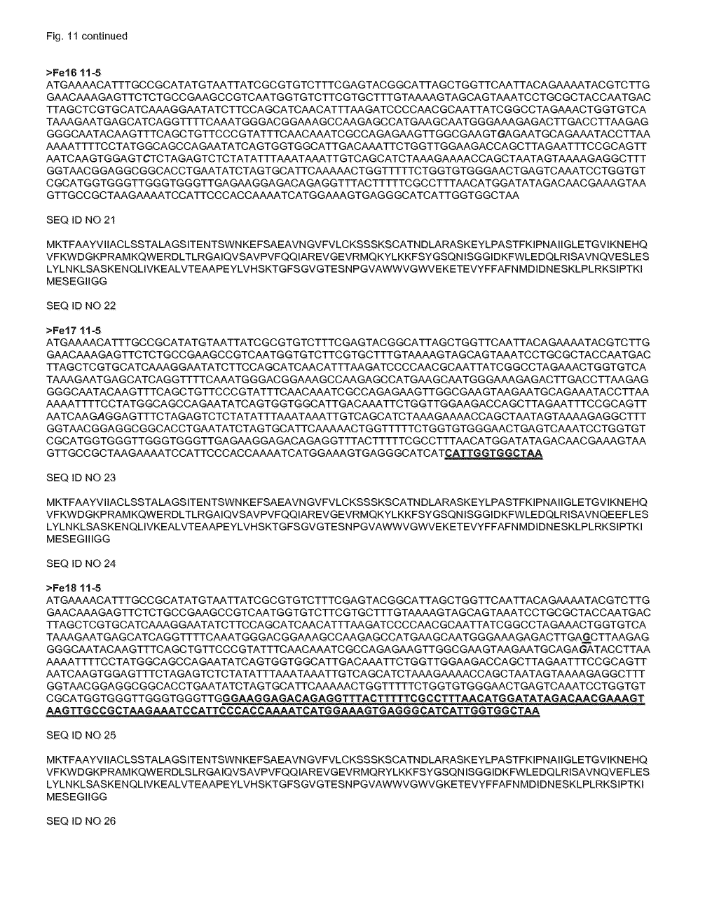

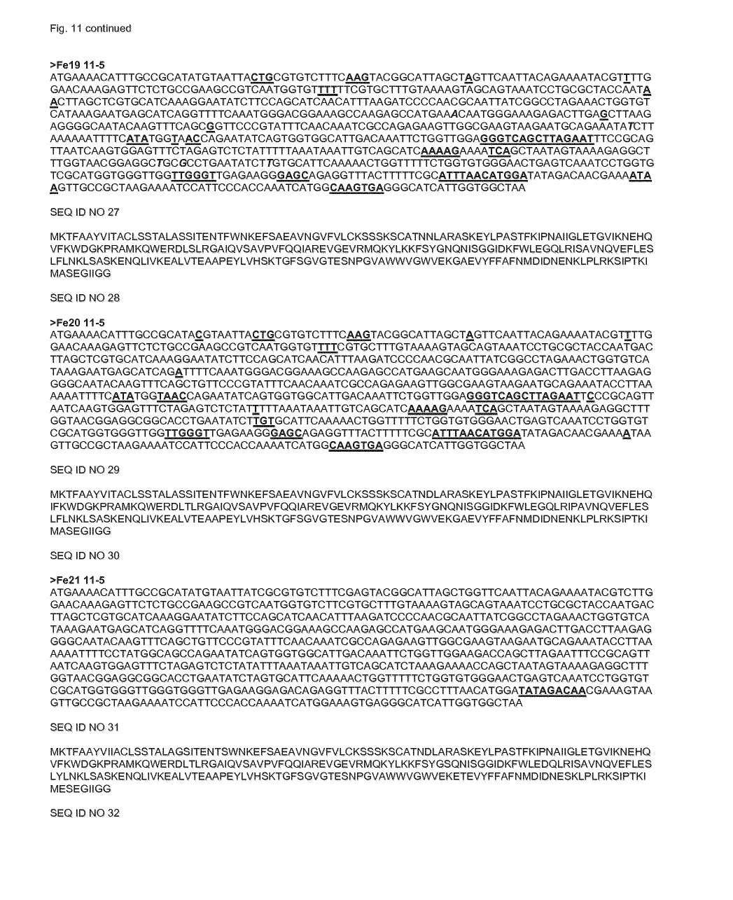

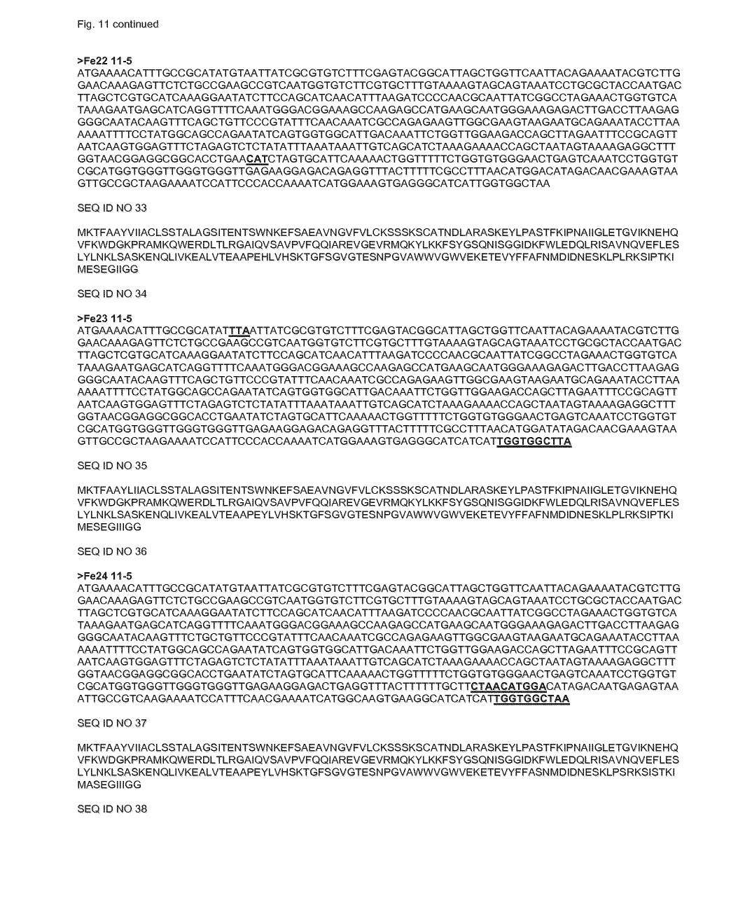

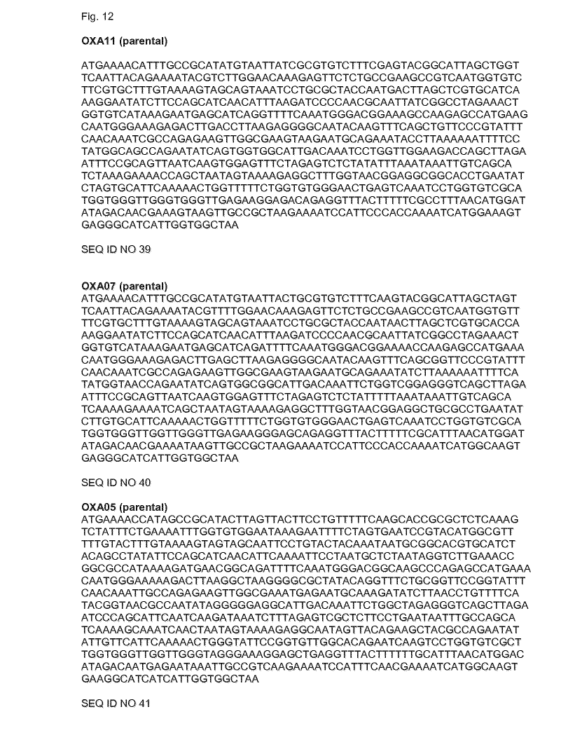

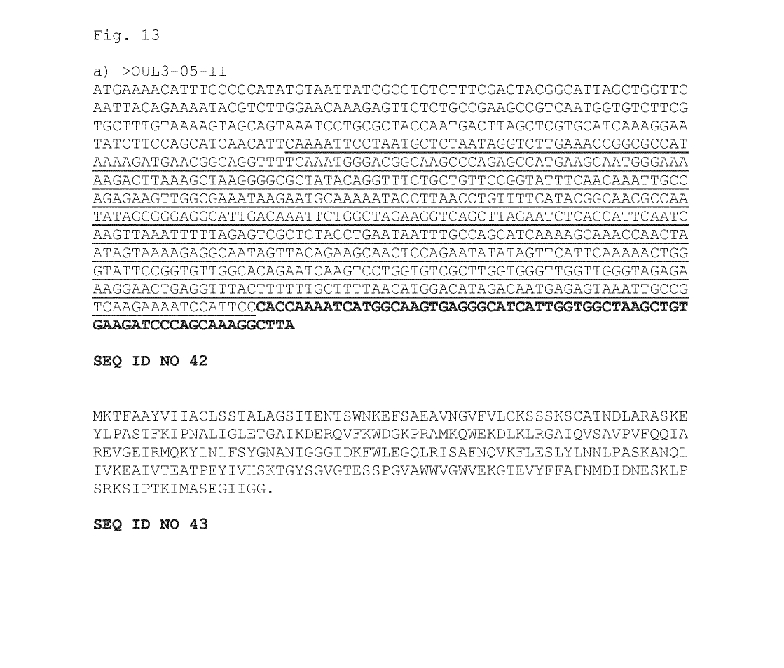

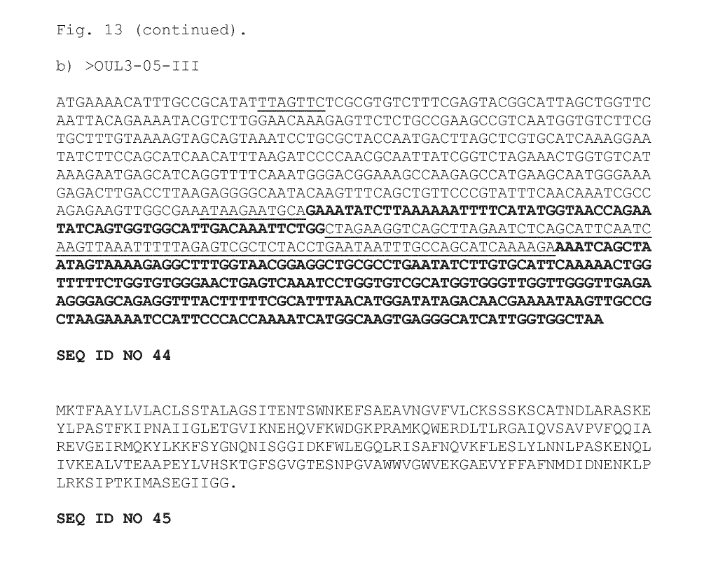

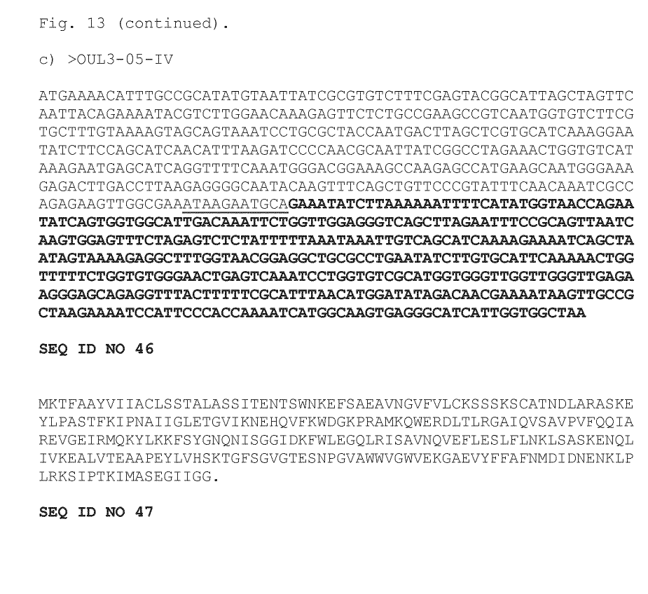

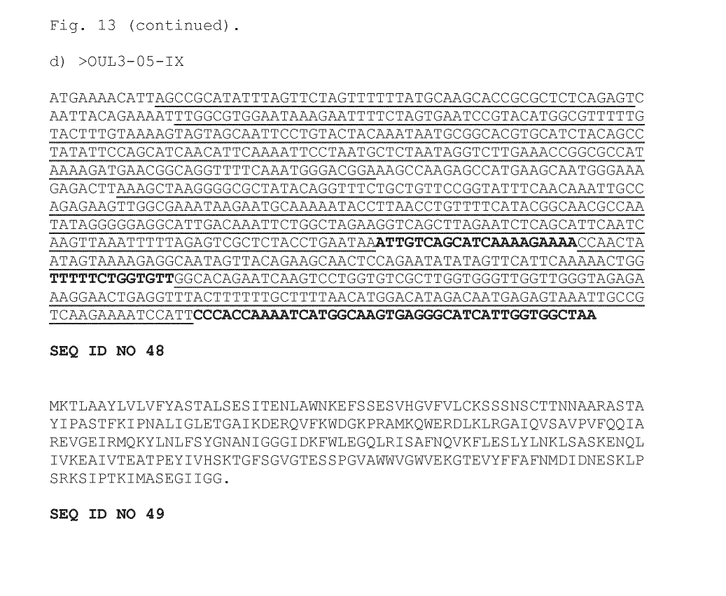

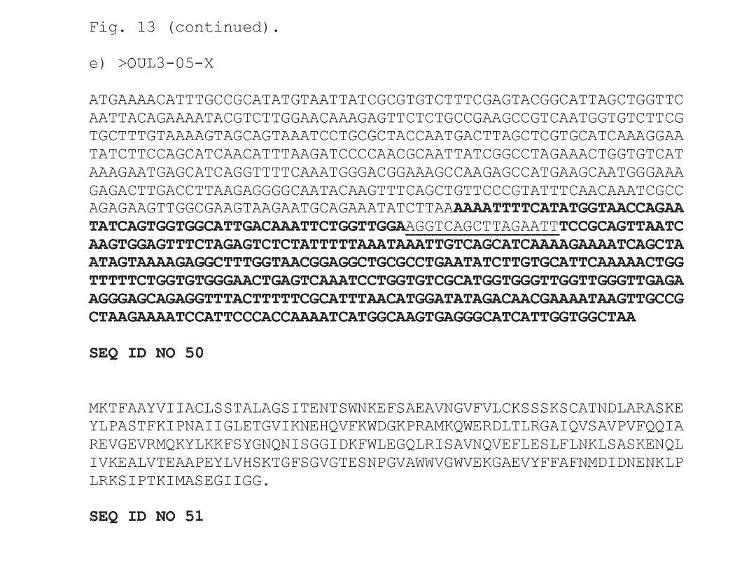

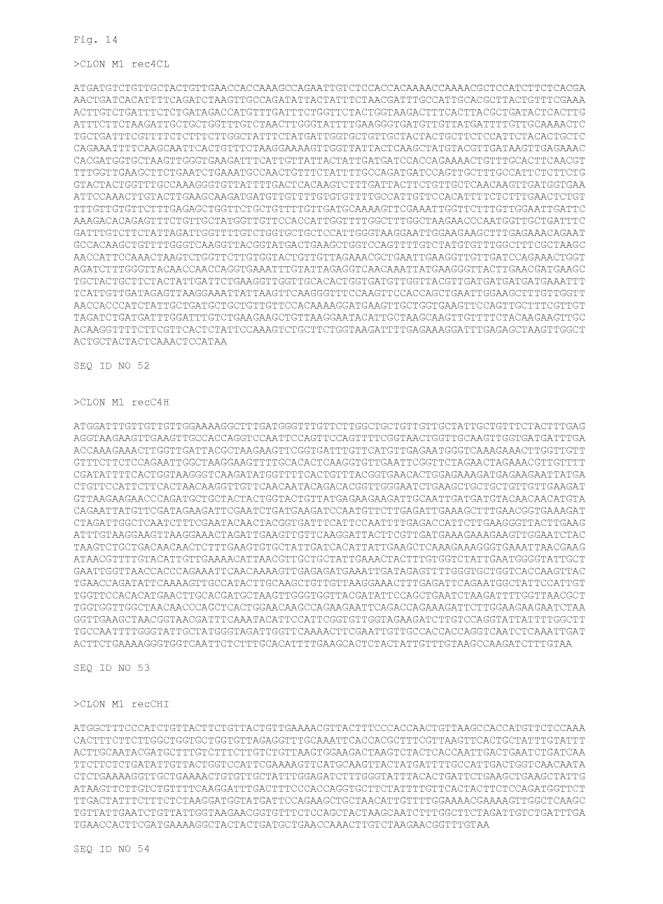

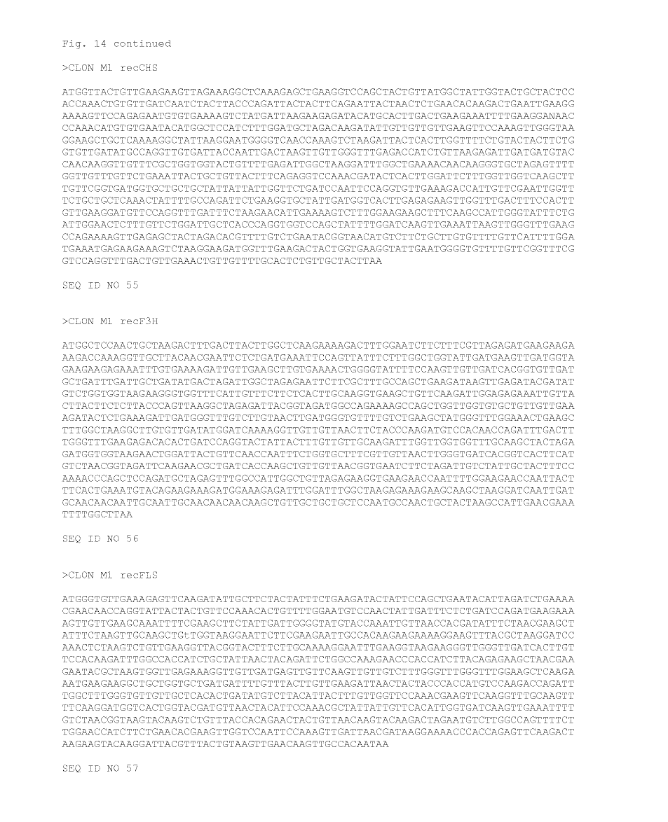

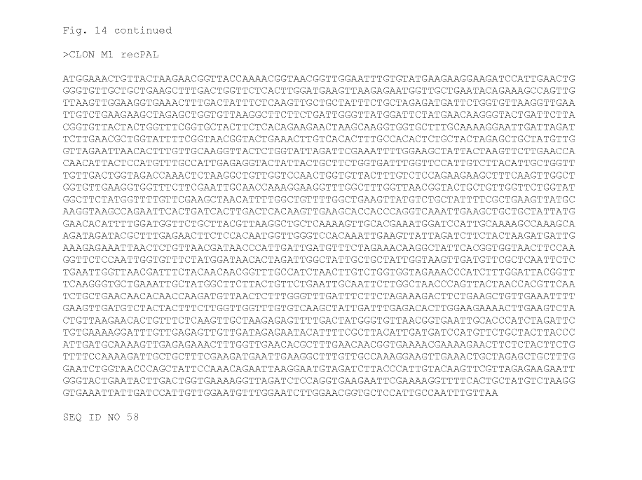

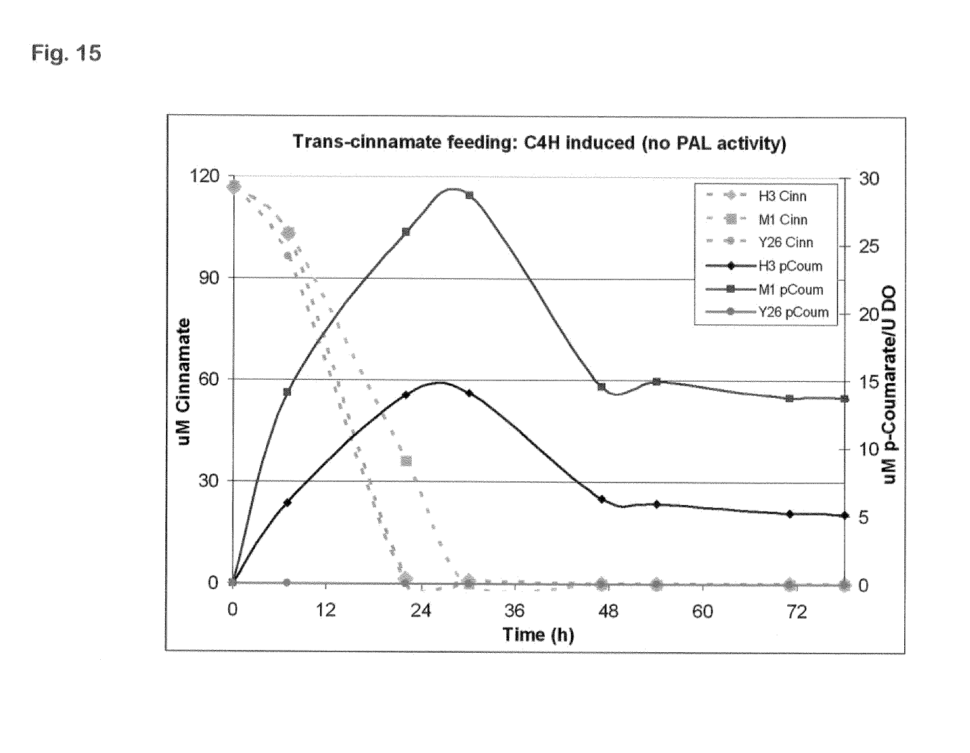

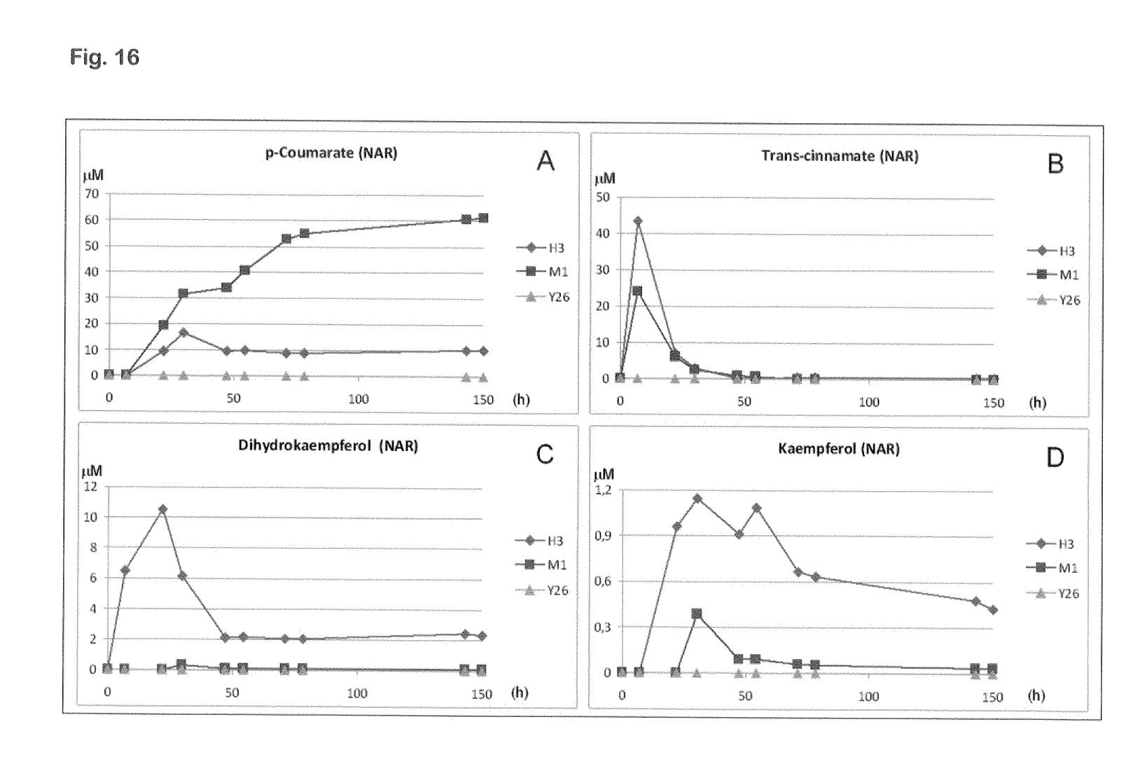

The homeologous genes A and B (homology of less than 99.5%) were recombined. As the marker sequences and the flanking target sequences are not homologous, recombination/assembly only occurred between genes A and B. As a consequence the hybrid/mosaic DNA contained recombined gene A and B, two markers and both flanking target sequences. The gene mosaic is integrated into the target locus on a target chromosome. Clones that have integrated the entire construct grew on appropriate media which is selective for both markers.

T 5′ and T 3′ correspond to the target sequences (homology of less than 99.5%) on the yeast genome (ca. 400 bp) addressing the homologous integration onto the chromosome site. M1 and M2 are the flanking markers for the double selection. Gene A and Gene B are related homeologous versions with a given degree of homology (less than 99.5%). Overlapping sequences correspond to the entire ORFs of both genes. After assembly by homeologous recombination in a MMR deficient yeast transformant, the double selection permits the isolation of recombinants.

FIG. 2: Recombination and Assembly of DNA by homeologous recombination

This figure shows a schematic presentation of a specific embodiment, wherein the cell is co-transformed with at least two genes, here DNA fragments A and B, which have homology of less than 99.5% on their overlapping fraction of 80 bp. Each DNA fragment was flanked by one selection marker.

Fragment A contained a flanking target sequence that corresponds to the 5′ end correct integration site on the chromosome and a hybridizing region that overlaps with fragment B, fragment B contained the flanking target sequence that corresponds to the 3′ integration site and a hybridizing region that overlaps with fragment A. Mismatch deficient yeast cells were transformed with the resulting fragments. The resulting transformants were plated on a medium, which is selective for both markers. Clones that can be selected for both markers were isolated, and the integrity of the assembled/integrated cluster, as well as the ORF's reconstitution of genes A and B were verified by molecular analysis of genomic DNA of selected recombinants.

T 5′ and T 3′ correspond to the target sequences (homology of less than 99.5%) on the yeast genome (ca. 400 bp) addressing the homologous integration onto the chromosome site. M1 and M2 are the flanking markers for the double selection. DNA fragments A and B can be either assembled to one gene, which can be traceable such as GFP, or can represent two genes which are assembled by this method. Overlapping sequences of all genes have homology of less than 99.5% (120 bp), permitting the reconstitution of the ORFs after assembly by homeologous recombination. Double selection permits the recombinant isolation and serves as primary verification of assembly.

FIG. 3: Recombination and Assembly of genes A, B and C

This figure shows the co-transformation of a further gene C, which has a sequence hybridizing with a flanking sequence of genes A and/or B to obtain assembly of said gene C to genes A and B.

T 5′ and T 3′ correspond to the target sequences (homology of less than 99.5%) on the yeast genome (ca. 400 bp) addressing the homologous integration onto the chromosome site. M1 and M2 are the flanking markers for the double selection. Gene A, Gene B and Gene C are related homeologous versions with a given degree of homology (less than 99.5%). Overlapping sequences correspond to the 5′ part and the 3′ part of the genes. The Gene B connects the flanking fragments and a new ORF ABC is reconstituted by sequence similarity. After assembly by homeologous recombination in a MMR deficient yeast transformant, the double selection permits the isolation of recombinants.

FIG. 4: Assembly of flavonoid pathways by fragments containing homeologous gene sequences

This figure shows the co-transformation of 8 fragments comprising the 8 genes for flavonoid production starting from phenylalanine. Each fragment hybridizes and recombines only in the region of the entire ORF of each parental gene (the homeologous or homologous sequences). By that way, the whole pathway is assembled in the DNA deficient repair yeast cell, and then integrated into the chromosome.

Tg 5′ and Tg 3′ correspond to the target sequences (homology of less than 99.5%) on the yeast genome (ca. 400 bp) triggering the homologous integration into the desired chromosome site. URA3 and HPH (hygromycin resistance) are the flanking markers enabling the double selection of the recombinant pathway. Genes CHI, F3H, PAL, CHS, C4H, FLS, and 4CL are related homeologous versions with a given degree of homology (less than 99.5%). Each gene possesses one promoter and one terminator sequence permitting their expression in yeast cells. Overlapping sequences correspond to entire ORF of the genes. After assembly of the fragments by homeologous recombination in a MMR deficient yeast transformant, a functional recombined complete pathway is reconstituted and the double selection permits the isolation of recombinants.

Plant sources of each gene are indicated with three letters following the name of the gene, also shown in three letters. The corresponding plant species are indicated at the left. Sequence identity between the homeologous version of genes is indicated in percent. The symbol * beside some fragments indicate that those were also used for homologous integration, meaning that gene sequences of overlapping fragments are 100% identical (wild type control, clone H3).

FIG. 5: Structure of the new recombinant flavonoid pathway and primers used to amplify each fragment for the assembly

This figure shows the final in vivo recombined structure of the integrated flavonoid pathway in DNA repair deficient yeast cells after transformation with synthetic DNA fragments as described in FIG. 4. Grey arrows (first and last arrows) correspond to the selection markers, black arrows indicate the specific genes for the flavonoid pathway, and white boxes represent the promoter and terminator sequences for each gene (see FIG. 4 for details). Also this figure lists the primers used to amplify each fragment as appear in FIG. 4. See table 6 for primer details and functions.

FIG. 6: Primers used to verify the assembly of the pathway and to amplify the recombinant genes for sequencing

As in FIG. 5, this figure shows the primers used to verify the assembly of the fragments after integration in yeast and to amplify the recombinant genes for sequencing analysis. See table 7 for primer details and functions.

FIG. 7: Mosaic sequences of a recombinant pathway obtained from the clone M1

7A: This picture shows the mosaic structure of the 7 recombinant genes obtained from the assembly and integration in yeast of the fragments containing the expression cassettes of the flavonoid pathway. Genomic DNA was extracted from the selected clones and specific primers used to amplify the recombinant flavonoid genes. White boxes correspond to the sequences derived from parent gene A (i.e. PA=CHI gene from Glycine max, also present in H3 [wild type control] clone). Black boxes correspond to the sequences derived from parent gene B (i.e. PB=CHI gene from Chrysanthemum sp.) The presence of more than one nucleotide exchange in the gene sequence of the M1 clone demonstrates the crossover event between parental genes as a result of the assembly (see sequences of FIG. 14 for details). 7B: references to gene names and plant sources for each parental gene used.

FIG. 8: Bacteriostatic effect of yeast supernatants derived from recombinant clones containing flavonoid pathways

This figure shows the inhibitory effect on E. coli cultured cells of yeast culture supernatants expressing flavonoid genes. 1/10 volume of induced supernatants of clones containing recombinant flavonoid pathways were added to 9/10 volumes of LB inoculated with E. coli TOP10 cells at an OD600 of 0.05. At several time points, OD was measured and the values obtained were compared to the corresponding control expressing no flavonoid genes. A strong inhibitory effect on E. coli growth was observed when supernatants of clones H3, M1, M7 and M8 were added. No remarkable effect was observed for clones M3 and M4, as well for negative control clone Y26 or by using the yeast media only.

FIG. 9: Quenching of DPH in the presence of flavonoids in yeast culture supernatants

This figure shows that compounds generated by clones expressing the recombinant flavonoid pathways are able to quench DPH, as it is known in the case of flavonoids. Yeast culture induced supernatants of clones containing recombinant flavonoid pathways were extracted with ethyl acetate and lyophilized. The pellets were recovered in 1/10 of initial volume of 70% ethanol. For each sample, 9 μl were added to 1 μl of 0.1 mM DPH (in DMSO) and mixed. Then, 5 μl were loaded on a Hybond-C membrane and the quenching of the samples was observed on an UV-transiluminator (inset A). The program Image-J was used to measure the signal intensity of the spots. Values were normalized to the signal obtained with the extract prepared from media only (no quenching). Naringenin (1 μM) was used as a control of maximum quenching. H3, M1, M4 and M7 clones revealed the higher values of quenching, and poor or none effect was observed with clones M2, M3 and Y26 (negative control).

FIG. 10: Sequences of gene and protein mosaics OXA11/OXA7 (SEQ ID NOs 1-14)

Nucleotide sequences of OXA7 origin are bold and underlined, mutation nucleotide sequences are bold and italic.

Clones were isolated by double selection and DNA used for amplification and sequencing. Only clearly readable sequences of both strands were used. Resulting chromatograms were aligned with a Clustal-like program.

FIG. 11: Sequences of gene and protein mosaics OXA11/OXA5 (SEQ ID NOs 15-38)

Nucleotide sequences of OXA5 origin are bold and underlined, mutation nucleotide sequences are bold and italic.

Clones were isolated by double selection and DNA used for amplification and sequencing. Only clearly readable sequences of both strands were used. Resulting chromatograms were aligned with a Clustal-like program.

FIG. 12: Sequences of parental genes OXA11 (P. aeruginosa, GI:296549, SEQ ID 39), OXA7 (E. coli, GI:516188, SEQ ID 40) and OXA5 (P. aeruginosa, GI:48856, SEQ ID 41)

FIG. 13: Sequences of clones comprising complex mosaic genes, corresponding to homeologous assembly OXA11/OXA5/OXA7

Sequences clones and results of respective protein annealing: FIG. 13a) OUL3-05-II (SEQ ID NOs 42 and 43), FIG. 13b) OUL3-05-III (SEQ ID NOs 44 and 45), FIG. 13c) OUL3-05-IV (SEQ ID NOs 46 and 47), FIG. 13d) OUL3-05-IX (SEQ ID NOs 48 and 49) and FIG. 13e) OUL3-05-X (SEQ ID NOs 50 and 51) of OXA11/OXA5/OXA7.

Nucleotide sequences of OXA 5 are bold and those corresponding to OXA 7 are underlined. Non bolded, non underlined sequences correspond to OXA 11.

FIG. 14: Sequences of recombinant clones of Example 3

Sequences clones and results provided as SEQ ID No: 52-58.

FIG. 15: Induced mosaic C4H enzyme of recombinant clone M1 is able to accumulate higher amounts of p-coumaric acid compared to control clone H3

This figure shows that under partial induction conditions and by adding cinnamic acid to the cultures, the supernatant of the clone M1 (mosaic flavonoid pathway) contained two times more p-coumaric acid than the control (no mosaic) H3 clone.

Cultures were grown in the presence of glucose, to repress PAL activity, without exogenous phenylalanine and methionine to induce C4H expression. Feeding with cinnamate was performed by adding 150 μM of the precursor to the medium. Aliquots of cultures were taken at different times and supernatants obtained by pelleting cells. Supernatants were extracted on SPE columns, and methanol extracts were separated by standards protocol using a C₁₈ HPLC column an acetonitrile/water gradient as described (4). Molar concentrations of p-coumaric acid and cinnamic acid in supernatants were calculated using area under the peaks prior calibriation with standards molecules. Values were normalized to the number of cells in the cultures. The production of p-coumaric acid is shown as continuous black lines. H3 control clone corresponds to black squares and mosaic M1 clone to black diamonds. Light grey lines shown the depletion of the feed precursor cinnamate.

FIG. 16: Different yields of intermediates and final flavonoid products in supernatants of control (no mosaic) and M1 (mosaic) clones during feeding with naringenin-chalcone

This figure shows 5 times higher amount of p-coumaric acid in M1 (black squares) supernatants than in H3 (black diamonds, inset A) when fully induced cultures are feed with 150 μM of naringenin-chalcone (NAR). Negative control values are traced with grey triangles. Supernatants were extracted on SPE columns, and methanol extracts were separated by standards protocol using a C₁₈ HPLC column and an acetonitrile/water gradient as described (4). The production and consumption of cinnamate (inset B) at 12 hours correlates with the production of dihydrokaempferol (inset C) at 12 hours, this last certainly deriving from the feed molecule naringenin. Final flavonoid product kaempferol appears just after (18 hours, inset D). M1 clone yields 3 times less amount of kampferol than control H3, however the mosaic clone consumes more of the precursor compared to H3. These differences suggest that the recombinogenic mosaicism results in improved production of final and intermediate products and show that recombinant pathways behave differently in same conditions of culture and feeding in the utilisation of precursor and/or intermediates.

### DETAILED DESCRIPTION OF THE INVENTION

Therefore, the present invention relates to the enzymatic evolution of variants of natural small aromatic molecules which was the first time possible through a novel and highly efficient method for in vivo recombination of homeologous DNA sequences, i.e. similar, but not identical sequences. Hereinafter the term homologous recombination, sometimes called homeologous recombination when homeologous sequences are recombined, refers to the recombination of sequences having a certain homology, which may or may not be identical. Unlike the conventional cloning approach that relies on site-specific digestion and ligation, homologous recombination aligns complementary sequences and enables the exchange between fragments. Recombinant mosaic genes, also called hybrid genes, are generated in the cell through hybridization of sequences having mismatched bases. By such an inventive mutagenesis method, it is possible to easily create diversity for suitable selections and redesign of polypeptides of interest in a time efficient manner.

Specifically, the invention employs the effective recombination and mosaic formation, diversification and assembly of diverse genes in a single step procedure, by the functional system of in vivo recombination.

The term “single step procedure” means that several process steps of engineering recombinants, like transformation of cells with a gene, the recombination of genes, generation of a mosaic gene and integration of a gene into the target genome, are technically performed in one method step. Thus, there would be no need of in vitro recombination of DNA carriers prior to in vivo recombination, or any repeating cycles of process steps, including those that employ meiosis. Advantageously, the use of meiotic yeast cells can be avoided.

The single step procedure according to the invention may even include the expression of such engineered recombinants by a host at the same time. Thereby no further manipulation would be necessary to obtain an expression product.

The term “gene mosaic” according to the invention means the combination of at least two different genes with at least one cross-over event. Specifically such a cross-over provides for the combination or mixing of DNA sequences. A gene mosaic may be created by intragenic mixing of gene(s), an intragenic gene mosaic, and/or gene assembly, which is understood as linking the genes, e.g. head-to-toe connection of at least two linear genes or parts of them, to obtain the gene assembly with intergenic cross-over, e.g. at an overlapping section, and composite genes stringed together, optionally with an overlap, further optionally assembly of genes with both, intragenic and intergenic cross over(s) or gene mosaic(s).

The term “cross-over” refers to recombination between genes at a site where two DNA strands can exchange genetic information, i.e. at least one nucleotide. The crossover process leads to offspring mosaic genes having different combinations of genes or sequences originating from the parent genes.

Alternatively, other repair mechanisms may be provided, which are not based on cross-over, e.g. nucleotide excision repair or non homologous end joining mechanisms comprising the recognition of incorrect nucleotides, excision and/or replacement after junction of strands.

The term “flanking target sequence” refers to regions of a nucleotide sequence that are complementary to the target of interest, such as a genomic target integration site, including a site of the gene(s) A and/or other gene(s) to be recombined, linear polynucleotides, linear or circular plasmids YAC's and the like. Due to a specific degree of complementation or homology, the flanking target sequence may hybridize with and integrate gene(s) into the target integration site.

The term “genome” of a cell refers to the entirety of an organism's hereditary information, represented by genes and non-coding sequences of DNA, either chromosomal or non-chromosomal genetic elements such as, linear polynucleotides, e.g. including the gene A and/or the other gene(s) to be recombined, viruses, self replicating carriers and vectors, plasmids, and transposable elements, including artificial chromosomes and the like. Artificial chromosomes are linear or circular DNA molecules that contain all the sequences necessary for stable maintenance upon introduction in a cell, where they behave similar to natural chromosomes and therefore are considered as part of the genome.

The term “homology” indicates that two or more nucleotide sequences have (to a certain degree, up to 100%) the same or conserved base pairs at a corresponding position. A homologous sequence, also called complementary, corresponding or matching sequence, as used according to the invention preferably is hybridising with the homologous counterpart sequence, e.g. has at least 30% sequence identity, but less than 99.5% sequence identity, possibly less than 95%, less than 90%, less than 85% or less than 80%, with a respective complementary sequence, with regard to a full-length native DNA sequence or a segment of a DNA sequence as disclosed herein. Preferably, a homologous sequence will have at least about 30% nucleotide sequence identity, preferably at least about 40% identity, more preferably at least about 50% identity, more preferably at least about 60% identity, more preferably at least about 70% identity, more preferably at least about 80% identity, more preferably at least about 90% identity, more preferably at least about 95% identity. Preferred ranges with upper and lower limits as cited above are within the range of 30% and 99.5% corresponding sequence identity. As used herein, the degree of identity always refers to the complementary sequences as well.

“Percent (%) identity” with respect to the nucleotide sequence of a gene is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

The term “anchoring” means the binding of a gene or gene mosaic to an integration sequence through a segment called “anchoring sequence” with partial or complete sequence homology, to enable the integration of such gene or gene mosaic into the integration site of a genome. Specifically the anchoring sequence can be a flanking target region homologous or partially homologous to an integration site of a genomic sequence. The preferred anchoring sequence has preferably at least about 70% sequence homology to a target integration site, more preferably at least 80%, 90%, 95% up to 99.5% or complete match with the hybridizing section of the genome.

The integration site may suitably be a defined locus on the host genome, where a high frequency of recombination events would occur. A preferred locus is, for example, the BUD31-HCM1 locus on chromosome III of S. cerevisiae. In general, any further loci on yeast chromosomes that show recombination at high frequencies but no change of cellular viability is preferred.

The term “expression” or “expression system” or “expression cassette” refers to nucleic acid molecules containing a desired coding sequence and control sequences in operable linkage, so that hosts transformed or transfected with these sequences are capable of producing the encoded proteins. In order to effect transformation, the expression system may be included on a vector; however, the relevant DNA may then also be integrated into the host chromosome.

The term “gene” shall also include DNA fragments of a gene, in particular those that are partial genes. A fragment can also contain several open reading frames, either repeats of the same ORF or different ORF's. The term shall specifically include nucleotide sequences, which are non-coding, e.g. untranscribed or untranslated sequences, or encoding polypeptides, in whole or in part.

The term “gene A” as used according to the invention shall mean any nucleotide sequence encoding a non-coding sequence or a polypeptide or polypeptides of interest. Gene A is characterized by being presented in the framework of a genetic construct, such as an expression cassette, a linear polynucleotide, a plasmid or vector, which preferably incorporates at least a marker sequence and has a single flanking target sequence, either at the 5′ end or 3′ end of gene A or the genetic construct. In the method according to the invention the gene A is typically a first gene in a series of genes to be recombined for gene mosaic formation. Gene A is homologous to another gene to be recombined, which is eventually either a variant of gene A, or any of genes B, C, D, E, F, G, H, etc., as the case may be. Thereby only one flanking target sequence per gene A is typically provided for the maximum fidelity purpose. Variants of gene A are called gene A1, A2, A3, etc., which have sequence homology to a certain extent, and optionally similar functional features. The term at least one gene A″ shall mean at least gene A and optionally variants of gene A.

The term “gene B” as used according to the invention shall mean any nucleotide sequence encoding a non-coding sequences or a polypeptide or polypeptides of interest, which is chosen for gene mosaic formation with another gene to be recombined, which is eventually either a gene A, a variant of gene B, or any of genes C, D, E, F, G, H, etc., as the case may be. Gene B is homologous to gene A or the other genes to a certain extent to enable mosaic formation with gene A or the other genes to be recombined. In the method according to the invention the gene B is typically the final gene in a series of genes to be recombined for gene mosaic formation. Gene B may be an integral part of the cell genome, or presented in the framework of a genetic construct, such as an expression cassette, a linear polynucleotide, a plasmid or vector, which preferably incorporates at least a marker sequence and has a single flanking target sequence, either at the 5′ end or 3′ end of gene B or the genetic construct, as a counterpart of the flanking target sequence of gene A, meaning at the opposite end of the gene. If the flanking target sequence of gene A is at the 5′ end of gene A, then the gene B would typically have its flanking target sequence on the 3′ end and vice versa. Thereby only one flanking target sequence per gene B is typically provided for the maximum fidelity purpose. Gene B may be a variant of gene A. Variants of gene B are called gene B1, B2, B3, etc., which have sequence homology to a certain extent, and optionally similar functional features. The term at least one gene B″ shall mean at least gene B and optionally variants of gene B.

The term “gene C” as used according to the invention shall mean any nucleotide sequence encoding a non-coding sequences or a polypeptide of interest. Gene C is characterized by being presented in the framework of a genetic construct, such as an expression cassette, a linear polynucleotide, a plasmid or vector, which optionally incorporates a marker sequence, and further characterised by a segment of its nucleotide sequence that is homologous to a sequence of gene A and/or gene B, a variant of gene C or eventually other genes D, E, F, G, H, etc, as the case may be. Gene C preferably has a single flanking target sequence, either at the 5′ end or 3′ end of gene C, or a flanking target sequence on both sides. Thereby gene C may partially or completely hybridize with gene A and/or the other genes to recombine, link and assemble the genes. In the method according to the invention the gene C is typically the second gene following gene A in a series of genes to be recombined for gene mosaic formation. Variants of gene C are called C1, C2, C3, etc, which have sequence homology to a certain extent, and optionally similar functional features.

A further gene D may be additionally recombined and assembled through hybridization of its nucleotide sequence or a segment of its nucleotide sequence that is homologous to a sequence of gene C, a variant of gene D or eventually other genes A, B, E, F, G, H, etc, as the case may be to provide the respective recombination and linkage. Gene D preferably has a single flanking target sequence, either at the 5′ end or 3′ end of gene D, or a flanking target sequence on both sides. In the method according to the invention the gene D is typically the next gene following gene C in a series of genes to be recombined for gene mosaic formation. Variants of gene D are called D1, D2, D3, etc, which have sequence homology to a certain extent, and optionally similar functional features.

A further gene E may be additionally recombined and assembled through a segment of its nucleotide sequence that is homologous to a sequence of gene D, a variant of gene E or eventually other genes A, B, C, F, G, H, etc, as the case may be to provide the respective recombination and linkage. Gene E preferably has a single flanking target sequence, either at the 5′ end or 3′ end of gene E, or a flanking target sequence on both sides. In the method according to the invention the gene E is typically the next gene following gene D in a series of genes to be recombined for gene mosaic formation. Variants of gene E are called E1, E2, E3, etc, which have sequence homology to a certain extent, and optionally similar functional features.

Further genes F, G, H, etc. may be used accordingly. The series of further genes is understood not to be limited by the number of alphabetical letters. The final chain of genes of interest would be obtained through linkage to the genes A and B to obtain the gene assembly at the integration site of the genome. The so assembled genes of interest may be operably linked to support the expression of the corresponding polypeptides of interest and metabolites, respectively. A specific method of assembly employs the combination of cassettes by in vivo recombination to assemble even a large number of DNA fragments to obtain desired DNA molecules of substantial size. Cassettes representing overlapping sequences are suitably designed to cover the entire desired sequence. In one embodiment the preferred overlaps are at least about 5 bp, preferably at least about 10 bp. In other embodiments, the overlaps may be at least 15, preferably at least 20 up to 1.000 bp.

In one preferred embodiment, some of the cassettes are designed to contain marker sequences that allow for identification. Typically marker sequences are located at sites that tolerate transposon insertions so as to minimize biological effects on the final desired nucleic acid sequence.

In a specific embodiment the host cell is capable of recombining or assembling even a large number of genes or DNA fragments of nucleic acids with overlapping sequences, e.g. at least 2, preferably at least 3, 4, 5, 6, 7, 8, 9, more preferably at least 10 genes or nucleic acid fragments in the host cell by co-transformation with a mixture of said genes or fragments and culturing said host to which the recombined or assembled sequences are transferred.

The genes or DNA fragments to be used according to the invention, either as a whole gene or in part, can either be double-stranded or single stranded. The double-stranded nucleic acid sequences are generally 300-20.000 base pairs and the single stranded fragments are generally shorter and can range from 40 to 10.000 nucleotides. For example, assemblies of as much as 2 Mb up to 500 Mb could be assembled in yeast.

Genomic sequences from a number of organisms are publicly available and can be used with the method according to the invention. These genomic sequences preferably include information obtained from different strains of the host cell or different species to provide homologous sequences having a specific diversity.

The initial genes used as substrates for recombination are a usually a collection of polynucleotides comprising variant forms of a gene. The variant forms show substantial sequence identity to each other sufficient to allow homologous recombination between substrates. The diversity between the polynucleotides can be natural, e.g., allelic or species variants, induced, e.g. error-prone PCR or error-prone recursive sequence recombination, or the result of in vitro recombination. Diversity can also result from resynthesizing genes encoding natural proteins with alternative codon usage. There should be at least sufficient diversity between substrates that recombination can generate more diverse products than there are starting materials. There must be at least two substrates differing in at least one or more positions. The degree of diversity depends on the length of the substrate being recombined and the extent of the functional change to be evolved. Diversity up to 69% of positions is typical.

According to the inventive method it is preferred that the genes A, B, C and further genes share a homology of at least 30% at least at a specific segment designed for hybridization, e.g. at an overlapping section, such as to obtain at least one cross-over at the overlap and optionally a gene assembly, which would include the full-length gene. The preferred homology percentage is at least 40%, more preferred at least 50%, more preferred at least 60%, more preferred at least 70%, more preferred at least 80%, more preferred at least 90%, even more preferred at least 95% up to less than 99.5%.

It may also be desirable simply to assemble, e.g. to string together and optionally mix such genes with gene variants, to diversify larger genes, e.g. members of an individual metabolic pathway or to assemble multiplicities of metabolic pathways according to this method. Metabolic pathways, which do not exist in nature, can be constructed in this manner. Thus, enzymes which are present in one organism that operate on a desired substrate produced by a different organism lacking such a downstream enzyme, can be encoded in the same organism by virtue of constructing the assembly of genes or partial genes to obtain recombined enzymes. Multiple enzymes can thus be included to construct complex metabolic pathways. This is advantageous if a cluster of polypeptides or partial polypeptides shall be arranged according to their biochemical function within the pathway. Exemplary gene pathways of interest are encoding enzymes for the synthesis of secondary metabolites of industrial interest, such as flavonols, macrolides, polyketides, etc.

In addition, combinatorial libraries can be prepared by mixing fragments, where one or more of the fragments are supplied with the same hybridizing sequences, but different intervening sequences encoding enzymes or other proteins.

Genetic pathways can be constructed in a combinatorial fashion such that each member in the combinatorial library has a different combination of gene variants. For example, a combinatorial library of variants can be constructed from individual DNA elements, where different fragments are recombined and assembled and wherein each of the different fragments has several variants. The recombination and assembly of a metabolic pathway may not need the presence of a marker sequence to prove the successful engineering. The expression of a metabolite in a desired way would already be indicative for the working example. The successful recombination and assembly of the metabolic pathway may, for example, be determined by the detection of the secondary metabolite in the cell culture medium.

Prokaryotic and eukaryotic host cells are both contemplated for use with the disclosed method, including bacterial host cells like E. coli or Bacillus sp, yeast host cells, such as S. cerevisiae, insect host cells, such as Spodooptera frugiperda or human host cells, such as HeLa and Jurkat.

Preferred host cells are haploid cells, such as from Candida sp, Pichia sp and Saccharomyces sp.

The inventive method would not use the sexual cycle or meiotic recombination. DNA fragments can be transformed into haploid cells. The transformants can be immediately streaked out on selective plates. The recombinants would then be isolated by PCR or other means, like gap repair.

The inventive process can be conducted in any wild-type or repair deficient prokaryotic or eukaryotic cells, including those with deficiency in nucleic acid repair, such as DNA or RNA repair. In wild-type cells, the suitable integration site is selected, which allows for homeologous recombination. The method according to the invention as carried out in wild-type cells preferably provides for the recombination of the genes, such as genes A and B, which have at least 80%, preferably at least 90% sequence identity. Though damaged and mismatched DNA is usually repaired and recombination is inhibited, it surprisingly turned out that homeologous recombination at the integration site is as well possible in such wild-type cells.

Mutations or modifications of the mismatch repair (MMR) system would enhance the frequency of recombination in the cells. Alternatively, other repair deficient systems may be used, such as completely or temporarily knock-outs of DNA repair genes rad1, recQ, which can enhance recombination.

DNA repair deficient cells are preferably used in the method according to the invention. As an example, mismatch repair can be completely or temporarily knocked out, or can be conditional or induced by addition of specific substrates to the cell culture medium, where the cells are cultured during or after targeted recombination is performed. Specifically, MMR deficiency of a cell can be achieved by any strategy that transiently or permanently impairs the mismatch repair, including the mutation of a gene involved in mismatch repair, treatment with UV light, treatment with chemicals, such as 2-aminopurine, inducible expression or repression of a gene involved in the mismatch repair, for example, via regulatable promoters, which would allow for a transient inactivation and activation.

Bacterial mismatch repair systems have been extensively investigated. In other systems, such as yeast, several genes have been identified whose products share homology with the bacterial mismatch repair proteins, e.g. analogues of the MutS protein, i.e. Msh1, Msh2p, Msh3p, Msh4, Msh5, Msh6p, and analogues of the MutL protein, i.e. Mlh1p, Mlh2p, Mlh3p, and Pms1 in S. cerevisiae.

Examples for preferred mismatch repair deficient cells are specific yeast cells, such as S. cerevisiae strains with defective or (temporarily) inactivated MSH2, e.g. engineered W303, BY, SK1 strains, such as MXY47 (W303 with disrupted MSH2) strain.

Further preferred systems of MMR are a selection of well-known bacterial strains, such as those described in U.S. Pat. No. 5,912,119, like strains defective for the enzymatic MutHLS mismatch repair system, e.g. of the mutS or mutL type, which is defective for the proteins MutS and MutL, which takes part in the recognition of the mismatches. Preferred strains are for example strains of S. Typhimurium using F⁻ mutL or recombinant E. Coli Hfr/S. Typhimurium F⁻ mutL.

Besides, other eukaryotic mismatch repair deficient cells, like HeLa and Jurkat cells are preferably used according to the invention.

The method according to the invention mainly employs marker assisted selection of a successful recombination product. The use of tools such as molecular markers or DNA fingerprinting can map the genes of interest. This allows screening of a large repertoire of cells to obtain a selection of cells that possess the trait of interest. The screening is based on the presence or absence of a certain gene.

The term “selection marker” as used according to the invention refers to protein-encoding or non-coding DNA sequences with provides for a mark upon successful integration. Specifically, the protein-encoding marker sequences are selected from the group of nutritional markers, pigment markers, antibiotic resistance markers, antibiotic sensitivity markers, fluorescent markers, knock-in markers, activator/binding domain markers and dominant recessive markers, colorimetric markers, and sequences encoding different subunits of an enzyme, which functions only if two or more subunits are expressed in the same cell. The term shall also refer to a traceable gene to be recombined that provides for the direct determination of the gene mosaic, without the need to use separate marker sequences.

A “nutritional marker” is a marker sequence that encodes a gene product which can compensate an auxotrophy of the cell and thus confer prototrophy on that auxotrophic cell. According to the present invention the term “auxotrophy” means that the cell must be grown in medium containing an essential nutrient that cannot be produced by the auxotrophic cell itself. The gene product of the nutritional marker gene promotes the synthesis of this essential nutrient missing in the auxotrophic cell. By successfully expressing the nutritional marker gene it is then not necessary to add this essential nutrient to the cultivation medium in which the cell is grown.

Preferred marker sequences are URA3, LEU2, CAN1, CYH2, TRP1, ADE1 and MET5.

A gene coding for a “pigment marker” is encoding a gene product, which is involved in the synthesis of a pigment which upon expression can stain the cell. Thereby rapid phenotypical detection of cells successfully expressing pigment markers is provided.

An “antibiotic resistance marker” is a gene encoding a gene product, which allows the cell to grow in the presence of antibiotics at a concentration where cells not expressing said product cannot grow.

An “antibiotic sensitivity marker” is a marker gene, wherein the gene product inhibits the growth of cells expressing said marker in the presence of an antibiotic.

A “knock-in” marker is understood as a nucleotide sequence that represents a missing link to a knock-out cell, thus causing the cell to grow upon successful recombination and operation. A knock-out cell is a genetically engineered cell, in which one or more genes have been turned off through a targeted mutation. Such missing genes may be suitably used as knock-in markers.

A “fluorescence marker” shall mean a nucleotide sequence encoding a fluorophore that is detectable by emitting the respective fluorescence signal. Cells may easily be sorted by well-known techniques of flow cytometry on the basis of differential fluorescent labeling.

The genes as used for diversification or recombination can be non-coding sequences or sequences encoding polypeptides or protein encoding sequences or parts or fragments thereof having sufficient sequence length for successful recombination events. More specifically, said genes have a minimum length of 3 bp, preferably at least 100 bp, more preferred at least 300 bp.

The preferred gene mosaics obtained according to the invention are of at least 3, preferably up to 20.000 base pairs, a preferred range would be 300-10.000 bp; particularly preferred are large DNA sequences of at least 500 bp or at least 1.000 bp.

Specifically preferred are gene mosaics that are characterized by at least 3 cross-over events per 700 base pairs, preferably at least 4 cross-overs per 700 base pairs, more preferred at least 5, 6 or 7 cross-overs per 700 base pairs or per 500 base pairs, which include the crossing of single nucleotides, or segments of at least 1, preferably at least 2, 3, 4, 5, 10, 20 up to larger nucleotide sequences.

According to the method of present invention not only odd but also an even number of recombination events can be obtained in one single recombined gene. This is a specific advantage over meiotic in vivo recombination.

Complex patterns of recombinant mosaicism can be obtained by the present method, reaching out high numbers of recombined sequence blocks of different length within one single molecule. Moreover, point-like replacement of nucleotides corresponding to one of the strand templates can be obtained as an important source of diversity respecting the frame of the open reading frames. Mosaicism and point-like exchange are not necessarily conservative at the protein level. Indeed, new amino acids with different polar properties can be generated after recombination, giving novel potential and enzymatic protein properties to the recombinant proteins derived by this method.

Preferably, the genes are non-coding sequences or protein-encoding sequences or parts of fragments thereof encoding enzymes or proteins of therapeutic or industrial applications. In the following the term “polypeptides” shall include peptides of interest having preferably at least 2 amino acids, preferably at least 3 polypeptides and proteins. The polypeptides of interest preferably are selected, but not limited to enzymes, transcription factors, transport proteins, signal peptides, receptors, hormones and growth factors. Respective recombinant variants resulting from the gene mosaics may trigger and catalyze the synthesis of new metabolites. Thus, it is the first time possible to effectively produce a large number of variants of natural small aromatic molecules by the metabolic evolution supporting the enzymatic synthesis process.

Specifically metabolites of aromatic amino acids, such as phenylalanine, tyrosine or and tryptophan, such as those produced by plants or yeast by enzyme activity, or any intermediates or derivatives may be produced in a novel way. The repertoire of enzyme variants thus leads to diverse metabolites formation, which is then screened for the desired structure and function. Those variants of natural small aromatic molecules have the advantage over the purely synthetic organic substances for their increased likelihood of possessing functional or biological activity.

Phe and Tyr are closely related. They contain a benzene ring which is additionally hydroxylated in tyrosine. Tyrosine is synthesized directly from the essential amino acid phenylalanine. Tryptophan contains a conjugated indole ring. These metabolic relations give rise to an intricate nutritional dependence.

In plants, the shikimate pathway produces the compound phenylalanine for the biosynthesis of phenylpropanoids. The hydroxycinnamates and esters produced by a combination of reductases, oxygenases, and transferases define the specific pattern of metabolites in an organ and depending on their development this profile is characteristic for each plant species. The initial three steps of the PP pathway are catalyzed by PAL, C4H and 4CL enzymes and provide the basis for all subsequent branches and resulting metabolites e.g.: flavonoids, lignins, phenylpropanoid esters, aurones, isoflavones, stilbenes, proanthocyanins, etc.

For example, PAL is known to catalyze the deamination of Phe to give cinnamic acid, which is the first step in the phenylpropanoid pathway and a regulation point between primary and secondary metabolism. Phenylpropanoid compounds are precursors to a range of phenolic compounds with many functions in nature, including lignin, flavonoids, isoflavonoids, coumarins and stilbenes.

Products of metabolic pathways are typically natural small molecules or variants thereof, e.g. differing in glycosylation, acylation, amination, hydroxylation or methylation with improved or new functions. These metabolites are suitably as fragrants or flavors or as therapeutic molecule (e.g. anti-infective or for the treatment of cancer).

The term “small aromatic molecules” as used herein shall refer to a range of small organic molecules having aromatic structure, wherein one or more CH groups may be replaced by heteroatoms, like N, O and S, including complex structures with different types and numbers of aromatic rings and various substituent groups.

Preferred examples of natural small aromatic molecules obtainable as metabolites are vanillin, coumaric acid, ferulic acid, lignin, 3-phenylpropanoid, flavonoids, anthocyanins.

Preferred examples having new functional properties are e.g. ethylvanillin (flavor), octylmethoxycinnamate and its esters (sunscreen), chalcone derivatives with increased antibacterial effect.

Intermediates of such metabolite production are sometimes useful as flavor or fragrance agents or as food ingredient.

Once synthesized as metabolites or intermediates of such metabolites by selected clones comprising the gene mosaic they are typically produced on the large scale by suitable expression systems, e.g. by microbial production, or by in vitro synthesis processes.

In a preferred embodiment of the invention the assembly of a mosaic gene, its recombination with a host genome, and further the expression of the mosaic gene to produce a recombinant polypeptide of interest or a metabolite of said host cell, is performed in a single step procedure.

In accordance with the present invention there may be conventional molecular biology, microbiology, and recombinant DNA techniques employed which are within the skill of the art. Such techniques are explained fully in the literature (9).

For in vivo recombination, the gene to be recombined with the genome or other genes is used to transfect the host using standard transfection techniques. In a suitable embodiment DNA providing an origin of replication is included in the construct. The origin of replication may be suitably selected by the skilled person. Depending on the nature of the genes, a supplemental origin of replication may not be required if sequences are already present with the genes or genome that are operable as origins of replication themselves.

Synthetic nucleic acid sequences or cassettes and subsets may be produced in the form of linear polynucleotides, plasmids, megaplasmids, synthetic or artificial chromosomes, such as plant, bacterial, mammalian or yeast artificial chromosomes.

A cell may be transformed by exogenous or heterologous DNA when such DNA has been introduced inside the cell. The transforming DNA may or may not be integrated, i.e. covalently linked into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA.

The diverse genes substrates may be incorporated into plasmids. The plasmids are often standard cloning vectors, e.g., bacterial multicopy plasmids. The substrates can be incorporated into the same or different plasmids. Often at least two different types of plasmid having different types of selectable markers are used to allow selection for cells containing at least two types of vector.

Plasmids containing diverse gene substrates are initially introduced into cells by any method (e.g., chemical transformation, natural competence, electroporation, biolistics, packaging into phage or viral systems). Often, the plasmids are present at or near saturating concentration (with respect to maximum transfection capacity) to increase the probability of more than one plasmid entering the same cell. The plasmids containing the various substrates can be transfected simultaneously or in multiple rounds. For example, in the latter approach cells can be transfected with a first aliquot of plasmid, transfectants selected and propagated, and then infected with a second aliquot of plasmid. Preferred plasmids are, for example, pUC and pBluscribe derivatives as pMXY9, pMXY12 and pMIX-LAM or YAC derivatives as YCp50.

The rate of evolution can be increased by allowing all gene substrates to participate in recombination. Such can be achieved by subjecting transfected cells to electroporation. The conditions for electroporation are the same as those conventionally used for introducing exogenous DNA into cells. The rate of evolution can also be increased by fusing cells to induce exchange of plasmids or chromosomes. Fusion can be induced by chemical agents, such as PEG, or viral proteins, such as influenza virus hemagglutinin, HSV-1 gB and gD. The rate of evolution can also be increased by use of mutator host cells (e.g., Mut L, S, D, T, H in bacteria, analogous mutants in yeast, and Ataxia telangiectasia human cell lines).

Cells bearing the recombined genes are subject to screening or selection for a desired function. For example, if the substrate being evolved contains a drug resistance gene, one would select for drug resistance.

Typically, in this inventive method of recombination, the final product of recombination that has acquired the desired phenotype differs from starting substrates at 0.1%-50% of positions and has evolved at a rate orders of magnitude in excess (e.g., by at least 10-fold, 100-fold, 1.000-fold, or 10.000 fold) of the rate of naturally acquired mutation. The final gene mosaic product may be transferred to another host more desirable for utilization of the shuffled DNA for production purposes.

In a preferred method according to the invention the host cell is displaying the gene mosaic on the cell surface using well-known cell display systems. By diversification through such hybridization a repertoire of gene variants is produced that can be suitably displayed to create a library of such variants.

Suitable display methods include yeast display and bacterial cell display. Particularly preferred libraries are yeast surface display libraries as used with many applications in protein engineering and library screening. Such libraries provide for the suitable selection of polypeptide variants with enhanced phenotypic properties relative to those of the wild-type polypeptide. Preferably cell-based selection methods are used, e.g. against surface-immobilized ligands. A commonly used selection technique comprises analyzing and comparing properties of the mutant polypeptide obtained from such library with properties of the wild-type polypeptide. Improved desirable properties would include a change of specificity or affinity of binding properties of a ligand polypeptide, which is capable of binding to a receptor. Polypeptide affinity maturation is a particularly preferred embodiment of the invention. Further desirable properties of a variant refer to stability, e.g. thermostability, pH stability, protease stability, solubility, yield or level of secretion of the recombinant polypeptide of interest.

A library obtained by the method according to the invention contains a high percentage of potential lead candidates of functional mosaic genes, which may be expressed in a functional ORF. The preferred library has at least 80% of the gene mosaics contained within a functional ORF, preferably at least 85%, at least 90%, even at least 95%. The library as provided according to the invention specifically is further characterized by the presence of the marker sequence indicating the high percentage of successful hybridization. According to the invention not only odd but also even numbers of mosaic patches can be obtained that increases the number of variants or library members in recombinant libraries produced by said method.

Usually libraries according to the invention comprise at least 10 variants of the gene mosaics, preferably at least 100, more preferred at least 1.000, more preferred at least 10⁴, more preferred at least 10⁵, more preferred at least 10⁶, more preferred at least 10⁷, more preferred at least 10⁸, more preferred at least 10⁹, more preferred at least 10¹⁰, more preferred at least 10¹¹, up to 10¹², even higher number are feasible.

The method according to the invention can provide a library containing at least 10² independent clones expressing functional variants of gene mosaics. According to the invention it is also provided a pool of preselected independent clones, which is e.g. affinity maturated, which pool comprises preferably at least 10, more preferably at least 100, more preferably at least 1.000, more preferably at least 10.000, even more than 100.000 independent clones. Those libraries, which contain the preselected pools, are preferred sources to select the high affinity variants according to the invention.

Libraries as used according to the invention preferably comprise at least 10² library members, more preferred at least 10³, more preferred at least 10⁴, more preferred at least 10⁵, more preferred at least 10⁶ library members, more preferred at least 10⁷, more preferred at least 10⁸, more preferred at least 10⁹, more preferred at least 10¹⁰, more preferred at least 10¹¹, up to 10¹² members of a library, preferably derived from a parent gene to engineer a new property to the corresponding polypeptide of interest.

Preferably the library is a yeast library and the yeast host cell preferably exhibits at the surface of the cell the polypeptide of interest having biological activity. Alternatively, the products are staying within the cell or are secreted out of the cell. The yeast host cell is preferably selected from the genera Saccharomyces, Pichia, Hansenula, Schizosaccharomyces, Kluyveromyces, Yarrowia and Candida. Most preferred, the host cell is Saccharomyces cerevisiae.

The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### EXAMPLES

### Example 1

### Description

In a first experimental set-up we used beta lactamase genes of the OXA class as substrate to be recombined. The advantage of the OXA genes lies in the fact that there are homeologous genes of different diversity (from 5-50%) available. These genes are therefore good candidates to test the limits of diversity of in vivo recombination. The genes are also easy to handle (about 800 bp length).

TABLE 1
Sequence identity of Oxa genes

| | Oxa 7 | Oxa 11 | Oxa 5 | Oxa 1 |
|---|---|---|---|---|
| Oxa 7 | 100% | | | |
| Oxa 11 | 95% | 100% | | |
| Oxa 5 | 77% | 78% | 100% | |
| Oxa 1 | 50% | 49% | 50% | 100% |

In the first experiment Oxa 11 was recombined with respectively Oxa 7 (95% identity), Oxa 5 (77% identity) and Oxa 1 (49% identity).

We used yeast strain BY47 derived from a strain collection (EUROSCARF) that contains knock outs of auxotrophic (-ura3, -leu2) marker genes and msh2. The gene defects in uracil and leucine biosynthetic pathway result in auxotrophy i.e. Uracil and Leucine have to be added to the growth media.

In a first step gene fragments were designed that contain on one hand the marker URA3 and OXA11 or on the other hand OXA 5/7/1 respectively with the other marker LEU2. Adjacent to the 5′ end of the URA-OXA11 fragment a DNA fragment of about 400 bp was inserted (5′ Flanking target sequence) that corresponds to the 5′ insertion site in the BUD 31 region of the yeast chromosome. At the 3′ end of the OXA 5/7/1 a DNA fragment of about 400 bp (3′ flanking target sequence) corresponding to the adjacent 3′ site on the chromosome (s. FIG. 3). All fragments were synthesized according to standard protocols at Geneart (Germany).

The synthesized fragments were amplified by PCR and used for transformation.

The URA3-OXA 11 fragment and one of the other OXA-LEU2 fragments were transformed into wild-type (diploid BY26240, Euroscarf) and mismatch deficient strains (haploid a-mater BY06240, msh2-, Euroscarf). The transformation protocol was according to Gietz (10). The transformants were plated on plates containing selective media for the selection on the appropriate markers (no uracil, leucine). After 72 hours colonies could be observed.

TABLE 2
Number of clones obtained after transformation/selection

| | Oxa11/ | Oxa11/ | Oxa11/ | Oxa11/ |
|---|---|---|---|---|
| Yeast/trafo | Oxa11 (1) | Oxa07 (2) | Oxa05 (3) | Oxa1 (4) |
| BY26240 | 10⁶ (5) | <10 | 0 | ND |
| (diploid msh wt) | | | | |
| BY06240 | 5 × 10⁴ | 5 × 10³ | 10³ | ND |
| (haploid Δmsh2) | | | | |

(1) Homologous control
(2) 5% of divergence at DNA level
(3) 23% of divergence at DNA level
(4) 51% of divergence at DNA level
(5) Estimated cpu number per ml of transformation mix and μg of DNA on selective media (-ura -leu)
ND = no colony detected

A total of 48 colonies issued from BY06240 transformation were isolated and colony PCR performed (lysis and Herculase PCR based on Cha and Thilly protocol (11). Different PCR reactions are performed to verify the correct insertion of the fragments into the target region. 37 clones out of 48 showed correct insertion profiles. From these 37, 31 gave clear and exploitable amplification products for sequencing. The reaction that uses two specific primers flanking the Oxa ORFs only permits the amplification of true recombinants if OXA sequences were actually assembled. Additionally, the obtained product is a correct substrate for direct sequencing. Thus, the positive amplification products were sequenced (GATC).

Results of Sequencing

24 clones out of 31 (those with the clearer positive amplification signals) were sequenced. They corresponded to: homologous control Oxa11/Oxa11 (SEQ ID NO 39), homologous control Oxa07/Oxa07 (SEQ ID NO. 40), homologous control Oxa05/Oxa05 (SEQ ID NO 41) fe02 to fe06, fe09 and fe11: Oxa11/Oxa07 (SEQ ID NO. 1 to SEQ ID NO. 14) fe09 and fe13, fe14, fe16 to fe24: Oxa11/Oxa5 (SEQ ID NO. 15 to SEQ ID NO. 38).

For sequencing results of all of the clones see FIGS. 10 and 11 and SEQ ID NOs 1 to 38.

For DNA annealing of Oxa11/Oxa07 clones see FIG. 10, SEQ ID NOs. 1, 3, 5, 7, 9, 11 and 13.

For DNA annealing of Oxa11/Oxa05 clones see FIG. 11, SEQ ID NOs. 15, 17, 19, 21, 23, 25, 27, 29, 31, 33, 35, and 37)

For protein annealing of OXA11/Oxa07 see FIG. 10, SEQ ID NOs. 2, 4, 6, 8, 10, 12 and 14.

For protein annealing of Oxa11/Oxa05 see FIG. 11, SEQ ID NOs. 16, 18, 20, 22, 24, 26, 28, 30, 32, 34, 36 and 38.

### Example 2

### Description

As a second alternative to generate libraries of complex mosaic genes, three different but related gene sequences were assembled and recombined. As in example 1, OXA gene sequences were used for their assembly in MMR deficient yeast. As showed in FIG. 3, the principle of mosaic generation is based on the usage of respectively truncated sequences of OXA 11 (gene A) and OXA 7 (gene B) that hybridize with the entire ORF of OXA 5 (gene C). Thus, only assembled and integrated cassettes A-B-C sharing the auxotrophic markers will be selected after transformation.

As in example 1 we used yeast strain BY47 derived from a strain collection (EUROSCARF) that contains knock outs of auxotrophic (-ura3, -leu2) marker genes and a deletion of msh2. The gene defects in uracil and leucine biosynthetic pathway result in auxotrophy: i.e. uracil and leucine have to be added to the growth media.

New gene fragments containing truncated genes A and B were obtained by specific PCR from the already described fragments in the example 1: URA-Oxa11 (reverse primer annealing on nucleotides 386-406 of OXA11 ORF) and OXA7-Leu (forward primer annealing on nucleotides 421-441 of OXA 7 ORF). The entire ORF of OXA 5 gene was obtained by PCR from fragment OXA5-Leu. The fragment END-Leu was used as in example 1. Purified PCR fragments were used for transformation.

The transformation protocol was according to Gietz (10). The transformants were plated on plates containing selective media for the selection on the appropriate markers (no uracil, leucine). After 72 hours colonies could be observed.

TABLE 3
Number of clones obtained after transformation/selection

| | Oxa11/Oxa5/ | Oxa11/ |
|---|---|---|
| Yeast/trafo | Oxa7 (1) | Oxa07 (2) |
| BY26240 | <10¹ (3) | ND (5) |
| (diploid | | |
| msh2 wt) | | |
| BY06240 | 1.4 × 10⁴ (4) | <5 |
| (haploid | | |
| Δmsh2) | | |

(1) Three OXA sequences to assemble
(2) Middle sequence OXA5 is missing (negative control)
(3) Homeologous recombination background in MMR proficient yeast
(4) Homeologous recombination background in MMR deficient yeast
(5) ND = no colony detected

A total of 8 colonies issued from BY06240 transformation were randomly isolated and colony PCR performed (lysis and Herculase PCR based on Cha and Thilly protocol (11). Different PCR reactions were performed to verify the correct insertion of the fragments into the target region. 7 clones out of 8 showed correct insertion profiles. From these 7 gave clear and exploitable amplification products for sequencing. The reaction that uses two specific primers flanking the Oxa ORFs only permits the amplification of true recombinants if OXA sequences were actually assembled. Additionally, the obtained product is a correct substrate for direct sequencing. Thus, the positive amplification products were sequenced (GATC).

Results of Sequencing

7 clones out of 8 (those with the clearer positive amplification signals) were sequenced 5 exploitable sequences were obtained. They corresponded all to homeologous assembly OXA11/OXA5/OXA7 from clones OUL3-05-II, OUL3-05-III, OUL3-05-IV, OUL3-05-IX and OUL3-05-X.

For sequencing results of all of the clones and protein annealing see sequences of selected clones (FIG. 13): OUL3-05-II (SEQ ID NOs 42 and 43), OUL3-05-III (SEQ ID NOs 44 and 45), OUL3-05-IV (SEQ ID NOs 46 and 47), OUL3-05-IX (SEQ ID NOs 48 and 49) and OUL3-05-X (SEQ ID NOs 50 and 51) of OXA11/OXA5/OXA7.

### DISCUSSION

This simple transformation method of mitotic MMR deficient cells with divergent sequences as templates for the assembly by the cell and generation of diversity by in vivo recombination has been proven.

Complex patterns of recombinant mosaicism have been obtained by the method described in example 1, reaching out at least 17 patches of different length into one single molecule of 800 bp (i.e. clones fe19 (SEQ ID NO 27) and fe20 (SEQ ID NO. 28). Recombination events seem to take place all the long of the sequences.

Moreover, point-like replacement of nucleotide corresponding to one of the strand templates were observed as an important source of diversity respecting the frame of the ORFs (i. e. clones fe19 (SEQ ID NO. 27) and fe20 (SEQ ID NO. 29).

In addition, this recombination method produced mosaics from more than two related genes as shown in the example 2 by using sequences from three related genes (OXA 11, OXA 7 and OXA 5) at the same time (i.e. clones OUL3-05-III and OUL3-05-IX). This is a highly efficient way to recombine regions of interest from several genes, and represents a new source of divergence based on the generation of mosaic genes libraries in vivo.

None of the recombinant clones yielded truncated protein products as verified by in silico analysis of translated DNAs.

Only 1 clone (fe15) out of 21 showed a parental profile (data not shown).

Mosaicism and point-like exchange are not necessarily conservative at the protein level. Indeed, new amino acids with different polar properties were generated after recombination, giving novel potential and enzymatic protein properties to the recombinant muteins (i.e. clones fe19 (SEQ ID NO. 27) and fe20 (SEQ ID NO. 29)

One very attractive trait of the recombinant generation by this approach making recombinant libraries richer is the fact that not only odd but also even number of recombination events could be obtained (i.e. fe06 (SEQ ID NO 7), fe11 (SEQ ID NO 13), fe13 (SEQ ID NO 17), fe19 (SEQ ID NO 27), compared to the meiotic recombination approach, by which only odd events could be represented into the library.

Some point mutations, not related to parental templates, were observed in a few numbers of sequences (i.e. fe16 (SEQ ID NO 21) and fe17 (SEQ ID NO 23). In all those cases, the mutations didn't change the reading frame of the resulting ORFs.

### Example 3

### Description

The flavonoid synthesis starts as all phenylpropanoids with phenylalanine. Seven enzymes are required for the conversion of L-phenylalanine to flavonol. Phenylalanine is converted to coumarate-CoA by the successive action of the enzymes phenylalanine lyase (PAL), cinnamate-4-hydrolase (C4H) and 4-coumaroyl-CoA ligase (4CL). The coumarate-CoA is a key branching point for the biosynthesis of different polyphenols. As an example, the coumarate-CoA is the precursor of a reaction cascade in which chalcone synthase (CHS), chalcone isomerase (CHI), flavanone 3-hydroxylase (F3H) and flavanol synthase (FLS) are involved. The NADP-cytochrome P450 oxyreductase (CPR) should be present for the catalysis of C4H and F3′H enzymes. Intermediate metabolites of the pathway serve as new substrates for a wide spectrum of flavonoids. Many plant genes of this metabolic pathway are characterized (12, 13, and references therein).

Most of these genes can be functionally expressed in yeast. However, neither expression level nor activity or stability has been described. Therefore we have opted for an inducible expression system to determine the biological activities as well as to analyse metabolic flux. Primary and/or high throughput screening methods were developed thereby using supernatants of the transformed cells (i.e. after assembly and recombination). Most of the flavonoid genes have homeologue counterparts, ranging from 95 to 70% in DNA sequence similarity.

Computational analysis has allowed designing 8 fragments containing the 8 genes of the Flavonoid pathway, as well as their homeologous recombination partners. The fragments, the ORF's as well as the upstream and downstream sequences are shown in FIG. 4 (for details on the sources of the sequences, see tables 4 and 5, for the amplification of each fragment, see FIG. 5B and table 6). In contrast to before mentioned references we have decided to assemble and recombine seven genes of the pathway in a 20 kb DNA stretch. The first and last fragment additionally contained target sequences for the integration of the pathway into the genome as shown in example 2. In this first experiment two genes of each enzyme were assembled/recombined. In order to have a reference for functional testing and comparison, a wild-type pathway containing only the same parental version gene (PA) of each enzyme was assembled.

TABLE 4
Reference identities of the genes used in this example 3

| | | Reference | ORF length |
|---|---|---|---|
| Gene | Species | (NCBI nucleotide database) | (bp) |
| 4CL | Glycine max | FJ770469.1 GI:225194702 | 1689 |
| | Populus tremuloides | AF041049.1 GI:3258634 | 1713 |
| C4H | Glycine max | FJ770468.1 GI:225194700 | 1521 |
| | Petroselinum crispum | Q43033.1 GI:3915088 | 1521 |
| CHI | Glycine max | Q43033.1 GI:3915088 | 681 |
| | Chrysanthemum × morifolium | EF094934.2 GI:121485997 | 708 |
| CHS | Hypericum androsaemum | AAG30295.1 GI:11096319 | 1170 |
| | Petunia × hybrida | P08894.1 GI:116385 | 1170 |
| CPR | Populus trichocarpa × Populus deltoides | AF302497.1 GI:13183563 | 2139 |
| F3H | Glycine max | AF302497.1 GI:13183563 | 1128 |
| | Hordeum vulgare | X58138.1 GI:18975 | 1131 |
| FLS | Fragaria × ananassa | AAZ78661.1 GI:73623477 | 1008 |
| | Solanum tuberosum | FJ770475.1 GI:225194714 | 1059 |
| PAL | Petroselinum crispum | X81158.1 GI:534892 | 2157 |
| | Populus trichocarpa × Populus deltoides | L11747.1 GI:169453 | 2148 |
| URA3 | Kluyveromyces lactis (pJJH726: nt 1 to | AF298788.1 GI:11344892 | 2146 |
| | 2246) | | |
| HPH | Streptomyces hygroscopicus | FJ834447.1 GI:257218583 | 1654 |
| | (YCP80: nt 631 to 2281) | | |
| CPR | Populus trichocarpa × Populus deltoides | AF302497.1 GI:13183563 | 2139 |

TABLE 5
Reference identities of the upstream and downstream sequences
for the flavonoid genes used in the example 3

| | | Reference | Length |
|---|---|---|---|
| | Species | (NCBI nucleotide database) | (bp) |
| Promoter | | | |
| pMET25Ski | Saccharomyces kudriavzevii | AACI01000009.1 GI:30995326 | 308 |
| | (nt 32899 to 33206 [C]) | | |
| pMET2Ppx | Saccharomyces paradoxus | AABY01000028.1 GI:29362583 | 474 |
| | (nt 14989 to 15458) | | |
| pGAL1/pGAL10 | pESC-URA (nt 2271-2934) | AF063585.2 GI:6446607 | 664 |
| pTRP1Sba | Saccharomyces bayanus | AACG02000058.1 GI:77693821 | 297 |
| | (nt 34484 to 34780) | | |
| pMET2Sby | Saccharomyces bayanus | AACG02000186.1 GI:77693693 | 479 |
| | (nt 4779 to 5247) | | |
| pADH1 | Saccharomyces cerevisiae | NC_001147.5 GI:84626310 | 1501 |
| | (nt 160595 to 162095 [C]) | | |
| pGDP | Saccharomyces cerevisiae | Part: BBa K124002 | 680 |
| Terminator | | | |
| tTPISce | Saccharomyces cerevisiae | J01366.1 GI:173007 | 243 |
| | (nt 1406 to 1649) | | |
| tPGKSce | Saccharomyces cerevisiae | J01342.1 GI:72143 | 286 |
| | (nt 1553 to 1839) | | |
| tADH1Sce | Saccharomyces cerevisiae | V01292.1 GI:3338 | 194 |
| | (nt 1798 to 1991) | | |
| tCYC1Sce | Saccharomyces cerevisiae | V01298.1 GI:3626 | 279 |
| | (nt 559 to 838) | | |

TABLE 6
Primers used for the amplification of the fragments used in example 3

| Primer | Sequence 5′−−>3′ | Function |
|---|---|---|
| AF1-Fw | CTGTGCTGTCTGCGCTGCATTC | Amplification Fragm. 1 |
| | SEQ ID 59 | |
| AF1-rv | TTACTTGTGAGATTGTGGATCAC | |
| | SEQ ID 60 | |
| AF2-Fw | ATGGCTTTCCCATCTGTTACT | Amplification Fragm. 2 |
| | SEQ ID 61 | |
| AF2-rv | ATGGCTCCAACTGCTAAGACT | |
| | SEQ ID 62 | |
| AF3-Fw | TTAAGCCAAAATTTCCTTCAATG | Amplification Fragm. 3 |
| | SEQ ID 63 | |
| AF3-rv | TTAACAAATTGGCAATGGAGAAC | |
| | SEQ ID 64 | |
| AF4-Fw | ATGGAAACTGTTACTAAGAACGGT | Amplification Fragm. 4 |
| | SEQ ID 65 | |
| AF4-rv | TTAAGTAGCAACAGAGTGCAAA | |
| | SEQ ID 66 | |
| AF5-Fw | ATGGTTACTGTTGAAGAATACAGAA | Amplification Fragm. 5 |
| | SEQ ID 67 | |
| AF5-rv | TTAGAAAGATCTTGGCTTAGCAAC | |
| | SEQ ID 68 | |
| AF6-Fw | ATGGATTTGTTGTTGTTGGAAA | Amplification Fragm. 6 |
| | SEQ ID 69 | |
| AF6-rv | TTATTGTGGCAACTTGTTCAAC | |
| | SEQ ID 70 | |
| AF7-Fw | ATGAAGACTATTCAAGGTCAATCT | Amplification Fragm. 7 |
| | SEQ ID 71 | |
| AF7-rv | TTATGGAGTTTGAGTAGCAGC | |
| | SEQ ID 72 | |
| AF8-Fw | ATGATTACTTTGGCTCCATCTTT | Amplification Fragm. 8 |
| | SEQ ID 73 | |
| AF8-rv | GCGCATGTGTCCGATCTTTG | |
| | SEQ ID 74 | |
| AF2m-Fw | ATGGCTTTCCCATCTGTTACT | Amplification Fragm. 2′ |
| | SEQ ID 75 | |
| AF2m-rv | ATGGCTCCAACTGCTAAGACT | |
| | SEQ ID 76 | |
| AF4m-Fw | ATGGAAACTGTTACTAAGAACGGT | Amplification Fragm. 4′ |
| | SEQ ID 77 | |
| AF4m-rv | TTAAGTAGCAACAGAGTGCAAA | |
| | SEQ ID 78 | |
| AF6m-Fw | ATGGATTTGTTGTTGTTGGAAA | Amplification Fragm. 6′ |
| | SEQ ID 79 | |
| AF6m-rv | TTATTGTGGCAACTTGTTCAAC | |
| | SEQ ID 80 | |
| AF7m-Fw | ATGAAGACTATTCAAGGTCAATCT | Amplification Fragm. 7′ |
| | SEQ ID 81 | |
| AF7m-rv | TTATGGAGTTTGAGTAGCAGC | |
| | SEQ ID 82 | |

TABLE 7
Primers used to analyze the assembly of the recombinant pathway and
to amplify the recombinant genes for sequencing analysis

| Primer | Sequence 5′−−>3′ | Function |
|---|---|---|
| BUD31-Fw | CACCAGCGCTCTAGATACAT | Insertion 5′ |
| | SEQ ID 83 | |
| URA3-rv | TGTGTTACCGATATCGGCGAAT | |
| | SEQ ID 84 | |
| CHI1-Fw | TCATTACCGCTTCCAAGTCTA | Amplification CHI |
| | SEQ ID 85 | |
| CHI2-rv | ACGCAGAATTTTCGAGTTATTA | |
| | SEQ ID 86 | |
| F3H2-Fw | AGAAGTGTCAACAACGTATCTAC | Amplification F3H |
| | SEQ ID 87 | |
| F3H3-rv | GGTGGTAATGCCATGTAATATGAT | |
| | SEQ ID 88 | |
| PAL3-Fw | TTCGGTTTGTATTACTTCTTATTCA | Amplification PAL |
| | SEQ ID 89 | |
| PAL4-rv | TACATGCGTACACGCGTCTGTA | |
| | SEQ ID 90 | |
| CHS4-Fw | CCAATTGGTTCAAGTCTCCAAAT | Amplification CHS |
| | SEQ ID 91 | |
| CHI2-rv | ACGCAGAATTTTCGAGTTATTA | |
| | SEQ ID 92 | |
| C4H5-Fw | CTACTGCCTAGCATCTTGCTAA | Amplification C4H |
| | SEQ ID 93 | |
| C4H6-rv | AATAGGGACCTAGACTTCAG | |
| | SEQ ID 94 | |
| FLS6-Fw | ACGCACACTACTCTCTAATG | Amplification FLS |
| | SEQ ID 95 | |
| FLS7-rv | CCCTTACAAGAACATTCACGAAAT | |
| | SEQ ID 96 | |
| 4CL7-Fw | AGACGGTAGGTATTGATTGTA | Amplification 4CL |
| | SEQ ID 97 | |
| 4CL8-rv | CGACCTCATGCTATACCTGAGA | |
| | SEQ ID 98 | |
| HYG-Fw | CTACTTCGAGCGGAGGCATC | Insertion 3′ |
| | SEQ ID 99 | |
| HcM1-rv | TCATTGCCTTCTCCACTCTC | |
| | SEQ ID 100 | |

Yeast transformation: The protocol for yeast transformation was slightly modified from Geitz and Woods (10). Cells were precultured in YPD media and then used to inoculate new rich media. They were harvested when OD600 reach out 0.6, the pellet washed twice and concentrated in 1/50 volume. Competent cells were added to the transformation PEG/LiAC/ssDNA mix with 500 ng of each fragment. Shortly, fragments (homologous and homeologous) were prepared in an equimolar mix and competent cells BY06 (msh2 deficient) were transformed. Additionally competent cells were transformed with no DNA (negative control). Selection of recombinant clones was performed on media without Ura and containing Hygromycin.

Analysis of Recombinants:

After 5 days clones transformed with the different fragments were observed on selection media. As expected, no clones were observed in the case of the no DNA control. 7 clones were randomly chosen for sequence analysis: 1 clone was selected from the transformation containing assembled wild-type genes, named H3. This clone served as wild-type control. 6 other clones resulting from the in vivo recombination of homeologous genes were also isolated and genomic DNA (gDNA) were prepared using the Wizard Genomic DNA purification kit (Promega). First, the correct integration of the cluster was analyzed by targeted PCRs from gDNA. In more than 90% of the selected clones amplified fragments corresponded to the expected length demonstrating correct integration. Then the 7 flavonoid genes of each of these clones were amplified with specific primers that also verified the correct assembly of the fragments. The amplification products were sequenced. (See assembled sequences of the recombinant clone M1 in FIG. 14, for details). The sequencing results demonstrated that we succeeded to reconstitute a complete flavonoid pathway in yeast by assembly, recombination and stable integration in the chromosome of the 8 genes involved in the pathway. The wild-type clone named H3 issued from assembly of 100% homologous genes, and the analysis of 6 recombinant clones revealed that the homeologous genes had recombined and assembled resulting in correct ORFs. We could not observe any frame shifts, deletions or insertions. The sequence analysis of the randomly isolated homeologous clones demonstrated that most genes of the flavonoid pathway were mosaics between their two parental versions.

Analysis of Yeast Clones Containing Recombinant Flavonoid's Pathways

a) Bacteriostatic Effect of Culture Supernatants of Recombinant Clones

Polyphenol metabolites such as flavonols or chalcones, have been described as compounds having a bacteriostatic or antimicrobial effect (14, 15, 16). This characteristic makes flavonoid derivatives and their precursors interesting candidates for drug development. The bacteriostatic effect can be screened by simple methods e.g. a photometric test for bacterial growth inhibition.

Supernatants of several of the afore mentioned clones were used to verify their bacteriostatic abilities when added to E. coli growing cultures (FIG. 8).

Yeast cultures were grown under inducing conditions (with galactose as the sole source of carbon, phenylalanine, and no methionine) for at least 72 hours. They were harvested by centrifugation and supernatants recovered. As controls, we used the Y06 strain (no Flavonol gene) cultured under the same conditions as the clones, and the media without yeast. 1/10 volume of these supernatants were added to LB media with an E. coli suspension (OD600=0.05). The bacterial cultures were grown at 30° C. and their cell densities were measured each 2 hours. As shown in FIG. 8, strong inhibitory effects on bacterial growth were observed in supernatants of clones HIII, M1, M7 and M8 when compared to the controls. Other clones produced a lower effect or no effect at all. Finally, these inhibitory factors are only present in clones with flavonoid genes and not in the control culture Y06 (no flavonoid genes) in this inducing condition.

Some flavonoid genes are controlled by inducible promoters such as GAL1/10 for PAL and F3H and MET 2/25 for CHI and C4H. In order to confirm the above inhibitory results, we cultured yeast clones in media with glucose and methionine to silence the inducible genes of the pathway and suppress the production of flavonoid metabolites. The non induced yeast cultures were treated as before and the supernatants used to analyze theirs inhibitory effects on E. coli cultures (data not shown). No inhibitory effect was observed for any of the recombinant flavonoid clones. Indeed, all of these supernatants essentially behave as the control Y06. Interestingly, when the clones are cultured in inducing conditions (with galactose, methionine and phenylalanine) the inhibitory effect on E. coli cultures was recovered (data not shown). These inhibitory effects are 5 fold higher in the case of clones HIII, M1 and M8, 2 fold or less for other clones, compared to the control Y06 in the same culture conditions.

b) Quenching of DPH by the Presence of Flavonoids and Precursors into Yeast Culture Supernatants Containing Recombinant Flavonoid Pathways

The test is based on the ability of the three-ringed molecules as flavonoids to quench the fluorescence of 1,6-diphenyl-1,3,5-hexatriene (DPH). This characteristic has been efficiently used to analyze flavonoid degrading microorganisms (17). The protocol was adapted to visualize the quenching of extracts containing flavonoids on a nylon membrane. Supernatants of yeast cultures grown in inducing conditions containing recombined flavonoid pathways were extracted with ethyl acetate and lyophilized. Then the pellets were recovered in 1/10 of initial volume of ethanol 70%. For each sample, 9 μl were added to 1 μl of DPH (0.1 mM in DMSO) and mixed. Then, 5 μl were loaded on a Hybond-C membrane and the quenching of the samples was observed on an UV-transiluminator (FIG. 8, inset A). The image-J program was used to measure the signal intensity of the spots and values were normalized to the signal obtained from extracts from media only (no quenching). Naringenin (1 μM) was used as a control of maximum quenching. The histogram of the values is shown (FIG. 8. inset B). Clearly, H3, M1, M4 and M7 clones revealed higher values of quenching, and poor or no effect was observed with clones M2, M3 and Y26 (negative control). These results are additional evidence that our yeast cells containing recombinant flavonoid pathways are synthesizing flavonoids and their intermediates.
c) HPLC Analysis of Yeast Recombinant Flavonoid Supernatants from Induced Cultures

Supernatants of some of the clones described to explore the inhibitory bacterial activity were extracted on SPE columns, and methanol extracts were separated by standard protocols using a C₁₈ HPLC column and an acetonitrile/water gradient as described (4). Additionally, peaks of interest were submitted to mass spectrometry by HPLC-MS by standard methods (4, 5) to identify the presence of flavonoid compounds in induced culture supernatants.

As already mentioned, wild type and negative control supernatants from cultures grown in partial inducing conditions were analyzed by HPLC. Supernatants of clones shown in FIG. 8 were further purified on SPE C18 Hydra columns following the indications of the supplier (Macherey-Nagel) and high performance liquid chromatography and mass analysis were performed as described (4, 5). Table 8 resumes the most important results obtained.

In order to determine the mass of selected peaks sample supernatants were extracted on SPE (C18) column and analyzed by HPLC-MS. 100 μl of extracts were first analysed by HPLC. It could be demonstrated that extracts from cells expressing flavonoid pathway genes contained new peaks when compared to a control containing no flavonoid genes (see table 8). Selected peaks were further analysed by mass spectrometry. Therefore, masses corresponding to the new peaks were fragmented and the resulting fragments were compared to fragments resulting from fragmentation of known flavonoid standards. Thus, caffeic acid, trans-cinnamic acid, p-coumaric acid, naringenin, dihydrokaempferol, kaempferol, and quercetin were identified, confirming the production of flavonoids in the yeast clone H3 and in mosaic clone M1, as an example.

As mentioned, more detailed analysis showed that H3 and some recombinant clones also accumulated flavonoid early precursors as p-coummarate and cinnamate. Surprisingly also caffeic acid, phloretic acid and styrene, three compounds not directly related to the flavonoid pathway were detected. It is reasonable to think that under certain conditions, yeast cells transformed with flavonoid genes are able to utilize precursors to produce new intermediates or that the recombination of different enzyme genes has resulted in enzymes with altered activity and substrate specificity. This could then reflect the diversity of molecules found in the metabolic compound library. Finally, several not yet known peaks were detected in the supernatants that are specific for clones containing the flavonoid recombinant pathways. (table 8).

TABLE 8
Specific peaks resolved by HPLC-MS from culture supernatants
of clones with recombinant flavonoid pathways

| | Clone | Clone | Y26 |
|---|---|---|---|
| Compound (mass) | H3 | M1 | (− cont) |
| Styrene (104) | + | + | − |
| Hydroxystyrene (120) | + | + | − |
| Cinnamate (148) | + | + | − |
| p-Coumarate (164) | + | + | − |
| Phloretic acid (166) | − | + | − |
| Caffeic acid (180) | + | − | − |
| Unknown (272) | + | + | − |
| Naringenin (272) | + | + | − |
| Kaempferol (285) | +(*) | +(*) | − |
| Dihydrokaempferol (288) | +(*) | +(*) | − |
| Unknown (295) | − | + | − |
| Quercetin (302) | + | − | − |

(*)Two haploids transformed cells were conjugated (mated) to obtain a diploid cell containing two identical pathways

d) Different Compound Profiles Between Homologous Flavonoid Recombinant and Mosaic Clones

CH4 Mosaic Enzyme and p-Coumaric Acid Production

Under total induction conditions and related to the same cell mass, it has been observed that the M1 clone is able to accumulate 3 times more coumaric acid than the homologous control H3 after separation by HPLC. This behaviour remains the same even if exogenous phenylalanine are omitted from the culture (cells use the endogenous amino acid). In both cases, cinnamic acid amount remains undetectable, suggesting that it is consumed at the same time that it is produced. When cell cultures are fed with 150 μM of cinnamic acid in absence of exogenous phenylalanine and without methionine added to the culture, there is twice as much coumarate in mosaic M1 clone than in H3 (FIG. 15). As expression of C4H enzymes in both clones is induced in the absence of methionine, M1 mosaic C4H enzyme in this culture is more active than its parental version in the one for the H3 clone.

F3H Mosaic Enzyme Converts More Precursor in Kaempferol than Parental H3

When induced H3 and M1 cell cultures were fed with 150 μM of naringenin-chalcone the same behaviour (higher p-coumarate production) for mosaic clone M1 was observed (FIG. 16A), with an apparently better conversion of cinnamate (FIG. 16B). At the same time, parental clone H3 was able to produce higher amounts of the flavonoid kaempferol even if the precursor dihydrokaempferol is not completely used (FIGS. 16C and D). On the other hand, mosaic M1 clone didn't accumulate dihydrokaempferol that seemed to have been completely converted to kaempferol (FIGS. 16C and D). The expression of the FLS enzyme is constitutive in both cases and seems to be the bottle neck for flavonoid production. F3H enzyme expression can be modulated in the presence of galactose. The fact that no accumulation of dihydrokaempferol is observed in mosaic clone M1 but in H3 under same culture media suggests that the mosaic enzyme has undergone a modification which is the cause for this different behaviour in dihydrokaempferol conversion and resulting kaempferol yields.

### DISCUSSION

The new one step method to assemble, recombine and express complex libraries of recombinant pathways has shown a remarkable efficacy. 14 related gene versions were used to generate mosaic pathways that could be modulated in their expression when assembled and integrated into DNA repair deficient cells. The process respects the structural integrity of the ORF permitting that recombinant forms of enzymes can be functionally expressed. In this specific case, we were able to identify flavonoids as final products of the pathway and their intermediates by modifying and inducing the corresponding genes. As the inducible expression of the recombinant pathways is functional, it gives us the possibility to generate intermediates and derivatives as well as final flavonoid products by simple media modification. HPLC supernatant analysis of induced recombinant cell cultures (mosaic pathway) has shown that production of certain molecules is higher compared to the control (no mosaic pathway), suggesting an amelioration of the catalyzing enzymes. Moreover, different molecules such as styrene, caffeic acid and phloretic acid were only found in the supernatants of recombinant clones. Finally, several not yet identified molecules containing flavonoid fragment signature were detected strengthening the concept that a complex phenylpropanoid compound library has been generated by this method. These results also open the possibility to exploit clones of the library in other pathways involved in the metabolism of aromatic compounds such as vanillin, styrene, amino acids, etc. Thus, the method can also be applied to other pathways and used in other organisms in which DNA repair mechanisms can be controlled in order to pro-mote homeologous recombination and gene cluster assembly to generate novel diversity.

### REFERENCES

1—Shao Z., Zhao H. and Zhao H. 2009. DNA assembler, an in vivo genetic method for rapid construction of biochemical pathways. Nucleic Acids Research 37(2):e16 Epu
2—Elefanti A., Begley C., Metcalf D., Barnett L., Köntgen F. and Robb L. 1998. Characterization of hematopoietic progenitor cells that express the transcription factor SCL, using a lacZ “knock-in” strategy. Proc. Natl. Acad. Sci. 95, 11897-11902
3—Koffas M., Leonard E., Yan Y. US patent published 5 Oct. 2010. Production of flavonoids by recombinant microorganisms
4—Naesby M et al. 2009. Yeast artificial chromosomes employed for random assembly of biosynthetic pathways and production of diverse compounds in Saccharomyces cerevisiae. Microbial Cell Factories. 8:45
5—Trantas E., Panopoulos N. and Ververidis F. 2009. Metabolic engineering of the complete pathway leading to heterologous biosynthesis of various flavonoids and stilbenoids in Saccharomyces cerevisiae. Metabolic Engineering. 11: 335-366
6—Radman M. and Rayssiguier C. WO patent 1990. In Vivo Recombination Of Partially Homologous DNA Sequences
7—Smith K. and Borts R. WO patent 2005. Generation de genes recombinants dans Saccharomyces cerevisiae.
8—Swers J., Kellogg B. and Wittrup K. 2004. Shuffled antibody libraries created by in vivo homologous recombination and yeast surface display. Nucleic Acids Research 32(3) e36
9—Sambrook J., Fritsch E. and Maniatis T. 1992. Molecular Cloning: A Laboratory Manual. ISBN-13: 978-0879693091
10—Gietz, R. and Woods R. 2002. Transformation of yeast by the LiAc/ss Carrier DNA/PEG method. Methods in Enzymology 350: 87-96
11—Cha R. and Thilly W. 1995. Specificity, Efficiency and fidelity of PCR, in PCR primer: A laboratory Manual, Dieffenbach and Dveksler eds., pp 37
12—Vogt T. 2010. Phenylpropanoids Biosynthesis. Mol Plant. Vol 3, 1: 2-20
13—Winkel-Shirley B. 2001. Flavonoid biosynthesis. A colorful model for genetics, biochemistry, cell biology, and biotechnology. Plant Physiol. 2:485-93
14—Oboh G. 2006. Antioxidant and antimicrobial properties of ethanolic extract of Ocimum gratissimum leaves. Journal Phar Tox, 1(1): 47-53
15—Karaman I, Gezegen H, Gürdere M, Dingil A, Ceylan M. 2010. Screening of biological activities of a series of chalcone derivatives against human pathogenic microorganisms. Chem Biodivers. 7(2): 400-8
16—Batovska D. and Todorova I. 2010. Trends in utilization of the pharmacological potential of chalcones. Curr Clin Pharmacol, 5(1):1-29, Review
17—Schoefer L., Braune A., Blaut M. 2001. A fluorescence quenching test for the detection of Flavonoid Transformation. FEMS Microbiology Letters 204: 277-280
18—Vogt T. 2010. Phenylpropanoids Biosynthesis. Mol Plant. Vol 3, 1: 2-20
19—Walton N., Mayer M., Narbad A. 2003. Molecules of Interest: Vanillin. Phytochemistry 63: 505-515
20—Sinha A., Sharma U., Sharma N. 2008. A comprehensive review on vanilla flavor: extraction, isolation and quantification of vanillin and others constituents. Int J Food Sci Nutr. 59: 299-326

1. A method for metabolic evolution of a variant of a natural small aromatic molecule product of a metabolic pathway by somatic in vivo assembly and recombination of said metabolic pathway employing a gene mosaic of at least one gene A, comprising:
a) in a single step procedure:
(i) transforming a cell with at least one gene A having a sequence homology of less than 99.5% to a second gene to be recombined that is an integral part of the cell genome or is presented in the framework of a genetic construct,
(ii) recombining said genes,
(iii) generating a gene mosaic of the genes at an integration site of a target genome, wherein said at least one gene A has a single flanking target sequence either at the 5′ end or 3′ end anchoring to the 5′ or 3′end of said integration site, and
(iv) recombining eventual further genes of said metabolic pathway, and
b) selecting clones comprising said gene mosaic and said eventual further genes capable of expressing said variant.

2. The method of claim 1, wherein the second gene is part of the genome of the cell.

3. A The method of claim 1, wherein the cell is co-transformed with at least one gene A and at least one gene B, wherein said single flanking target sequence of gene A is anchoring to the 5′end of an integration site on said target genome, and wherein gene B is linked to a single flanking target sequence anchoring to the 3′ end of the integration site.

4. The method of claim 1, wherein the cell is co-transformed with at least two different genes A1 and A2 and optionally with at least two different genes B1 and B2.

5. The method of claim 1, wherein at least one further gene C is co-transformed, wherein gene C has a sequence hybridizing with a sequence of gene A and/or with the second gene to obtain assembly of said further gene C to gene A and/or to the second gene.

6. The method of claim 1, wherein said gene A and/or the second gene is a non-coding sequence or a sequence coding for a polypeptide or for part of a polypeptide having an activity.

7. The method of claim 1, wherein at least two genes of said metabolic pathway are recombined and assembled.

8. The method of claim 7, wherein said genes are linear polynucleotides, preferably polynucleotides of between 300 and 20,000 base pairs.

9. The method of claim 1, wherein gene mosaics of from at least 3 and up to 20,000 base pairs, preferably with at least 3 cross-over events per 700 base pairs are obtained.

10. The method of claim 1, wherein the cell is a DNA repair deficient cell.

11. The method of claim 1, wherein the cell is a eukaryotic cell, preferably a fungal, mammalian or plant cell, or a prokaryotic cell.

12. The method of claim 1, wherein the natural small aromatic molecule is selected from the group consisting of a phenylpropanoid, a flavonoid, a flavanol, an anthocyanine, a lignin, a cyanidin, a chalcone, vanillin, and a naturally occurring derivative thereof.

13. The method of claim 1, wherein said variant is synthesized by recombinant enzyme variants.

14. A method of preparing a library of cells producing variants of natural small aromatic molecule products of a metabolic pathway, comprising engineering recombinant cells by somatic in vivo assembly and recombination of said metabolic pathway employing a gene mosaic of at least one gene A, which comprises:
a) in a single step procedure
(i) transforming a cell with at least one gene A having a sequence homology of less than 99.5% to a second gene to be recombined that is an integral part of the cell genome or presented in the framework of a genetic construct,
(ii) recombining said genes,
(iii) generating a gene mosaic of the genes at an integration site of a target genome, wherein said at least one gene A has a single flanking target sequence either at the 5′ end or 3′ end anchoring to the 5′ or 3′end of said integration site, and
(iv) recombining eventual further genes of said metabolic pathway, and
b) collecting clones comprising said gene mosaic and said eventual further genes to obtain a library capable of producing said variants.

15. A library of cells obtained by a method according to claim 14, comprising at least 10E3 different clones producing said variants.

16. The library of claim 15, wherein the library of cells comprises recombinant genes encoding a repertoire of metabolic pathways.

17. The library of claim 15, a wherein the library of cells comprises recombinant genes encoding a repertoire of synthesizing enzymes.

18. The method of claim 14, further comprising the step of producing a library of synthesizing recombinant enzymes obtained from the library.

19. A non-human organism that comprises a gene variant obtained from the library of claim 15.

20. The method of claim 14, further comprising the step of selecting a cell producing a desired variant of a natural small aromatic molecule from the library.

21. The method of claim 20, further comprising the step of determining the structure and function of said variant.

22. The method of claim 20, further comprising the step of producing said variant in a non-human recombinant host.

23. The method of claim 20, further comprising the step of synthetically producing said variant.

24. The method of claim 20, wherein said variant is a phenylpropanoid with a biological activity selected from the group consisting of antibacterial activity, antioxidative activity, fragrance activity and flavor activity.
